(19) United States
(12) Patent Application Publication
(10) Pub. No.: US 2003/0211995 A1
(43) Pub. Date: Nov. 13, 2003

(54) Methods and formulations for eradicating or alleviating staphylococcal nasal colonization using lysostaphin

(76) Inventors: John Fitzgerald Kokai-Kun, Frederick, MD (US); Scott Michael Walsh, Germantown, MD (US); James Jacob Mond, Silver Spring, MD (US); Tatyana Ivanovna Chanturiya, Rockville, MD (US)

Correspondence Address: Finnega, Henderson, Farabow,, Garrett & Dunner, L.L.P., 1300 I Street, N.W., Washington, DC, 20005-3315, US

(21) Appl. No.: 10/323,907

(22) Filed: Dec. 20, 2002

Related U.S. Application Data
(60) Provisional application No. 60/341,802, filed on Dec. 21, 2001.

(51) Int. Cl. A61K 38/17

(52) U.S. Cl. 514/12

(57) ABSTRACT

This invention provides lysostaphin intranasal compositions that can be administered to the anterior nares of those at risk for staphylococcal nasal colonization and subsequent infection and methods for their use.

### CROSS-REFERENCE TO RELATED APPLICATIONS

This application is based on and claims the benefit of U.S. Provisional Application S. No. 60/341,802, filed Dec. 21, 2001 (Attorney Docket No. 7787.6009). The entire disclosure of this provisional application is relied upon and incorporated by reference herein.

### INTRODUCTION

Staphylococcal infections are a significant cause of morbidity and mortality, particularly in settings such as hospitals, nursing homes, schools, and infirmaries. Patients particularly at risk include infants, the elderly, the immunocompromised, the immunosuppressed, those convalescing, and those with chronic conditions requiring frequent hospital stays. Further, the advent of multiple drug resistant strains of Staphylococcus aureus increases the concern and need for timely blocking and treatment of such infections. Indeed, the recent World Health Organization report entitled “Overcoming Astionicro Oral Resistance” detailed its concern that increasing levels of drug resistance are threatening to erode the medical advances of the recent decades. Among the issues raised are infections in hospitalized patients. In the United States alone, some 14,000 people are infected and die each year as a result of drug-resistant microbes acquired in hospitals. Around the world, as many as 60% of hospital-acquired infections are caused by drug-resistant microbes.

In infections caused by S. aureus, it appears that a principal ecological niche for S. aureus in humans is the anterior nares. Nasal carriage of staphylococci plays a key role in the epidemiology and pathogenesis of infection (13, 23, 34, 51, 68, 71, 72, 74). In healthy subjects, three patterns of S. aureus nasal carriage can be distinguished over time: approximately 20% of people are persistent carriers, approximately 60% are intermittent carriers, and approximately 20% apparently never carry S. aureus (34).

Nasal carriage of staphylococci is an important risk factor for contracting S. aureus infection. Patients at greatest risk are those undergoing inpatient or outpatient surgery, in the Intensive Care Unit (ICU), on continuous hemodialysis, with HIV infection, with AIDS, burn victims, people with diminished natural immunity from treatments or disease, chronically ill or debilitated patients, geriatric populations, infants with immature immune systems, and people with intravascular devices or other foreign bodies (13, 23, 25, 34, 35, 41, 51, 72, 74). In one study of ICU patients (19), it was found that on admission 166 of 752 (22%) of patients were S. aureus nasal carriers. The probability of developing a staphylococcal infection was significantly greater (p<0.0001, with a relative risk of 59.6) in these patients than in non-carriers. In 28 out of 30 cases of subsequent staphylococcal infection, researchers found the S. aureus strain colonizing the nares to be identical to the strain isolated from the infection. Even more strikingly, Mest et al. (46) showed that, of 19 patients who were admitted to the ICU with positive, nasal cultures for S. aureus, 5 (26%) subsequently developed staphylococcal infections as compared to only 6 S. aureus infections in a group of 465 patients (1.3%) negative for nasal carriage of staphylococci.

Chang et al. (12) studied 84 patients with cirrhosis admitted to a liver transplant unit. Overall, 39 (46%) were nasal carriers of S. aureus and 23% of these patients subsequently developed S. aureus infections as compared to only 4% of the non-carriers. A study of HIV patients (51) showed that 49% (114 of 296) of patients had at least one positive nasal culture for S. aureus. Thirty four percent of 201 patients were considered nasal carriers, with 38% of these being persistent carriers, and 62% intermittent carriers. Twenty-one episodes of S. aureus infection occurred in thirteen of these patients. Molecular strain typing indicated that, for six of seven infected patients, the strain of S. aureus isolated from the infected site was the same as that previously cultured from the nares. The nasal S. aureus carrier patients were significantly more likely to develop S. aureus infection (P=0.04; odds ratio, 3.6; attributable risk, 0.44). This finding led the authors to conclude that nasal carriage is an important risk factor for S. aureus infection in HIV patients (51).

As with many bacterial pathogens, antibiotic resistance in staphylococci is an ever-emerging problem. This problem extends even to nasal colonization by S. aureus where many strains found in studies of nasal colonization are antibiotic resistant. For example, methicillin resistant S. aureus (“MRSA”) is a well documented public health problem (23, 25, 46). In one study performed in a nursing home, 29% of the residents carried S. aureus in the nares and, of those isolates, 31% were MRSA (37). In a separate study of post operative intra-abdominal infection, it was concluded that MRSA may be a causative pathogen in postoperative intra-abdominal infection and that this may be related to nasal colonization (23).

After examination of the above cited literature, it is clear that nasal colonization by both antibiotic sensitive and antibiotic resistant S. aureus is a major health risk that needs to be addressed. Current technology indicates using Bactroban Nasal (2% mupirocin cream) to clear staphylococcal nasal colonization (22, 35, 41, 63, 72). Indeed, this is the only commercially available product specifically for the elimination of S. aureus nasal colonization. While other antibiotics not specifically formulated for nasal use have also been used with limited success as intranasal antimicrobial agents for eradicating S. aureus nasal colonization (26, 35, 63), Bactroban Nasal remains the most effective currently available treatment for S. aureus nasal colonization. Unfortunately, as is the case with methicillin, mupirocin resistant strains of S. aureus (MupRSA) are emerging in many different geographical areas (14, 18, 20, 40). Therefore, based on these considerations, there is a need in the art for an intervention which is immediate and directed to the mammalian nares.

### BRIEF DESCRIPTION OF THE INVENTION

This invention relates to formulations comprising lysostaphin for intranasal use (“lysostaphin intranasals”). Lysostaphin is an antibacterial enzyme first identified in a strain of Staphylococcus simulans (formerly known as S. staphylolyticus) in 1964. Lysostaphin is an endopeptidase capable of specifically cleaving the cross-linking pentaglycine bridges in the cell walls of staphylococci. Because the cell wall bridges of S. aureus contain a high proportion of pentaglycine, lysostaphin is highly effective in lysing S aureus, although activity against other species of staphylococci has been demonstrated (75). Lysostaphin does not require active bacterial growth to elicit its antibacterial effects. In contrast, β-lactams such as methicillin, exhibit antibacterial effects only on bacteria that are actively growing.

The lysostaphin present within the lysostaphin intranasal compositions of the invention may be isolated from natural bacterial sources; artificially generated recombinant forms of lysostaphin; active recombinant, enzymatic, or synthetic fragments of lysostaphin; or complete synthetic lysostaphin molecules capable of specifically cleaving the cross-linking pentaglycine bridges in the cell walls of staphylococci. This invention also relates to the administration of lysostaphin intranasals to the nares to alleviate or block staphylococcal nasal colonization. Those at risk for invasive disease as a consequence of staphylococcal nasal colonization include the very young, the very old, patients admitted to the hospital for in-patient or out-patient surgical procedures, patients suffering from various conditions that predispose them to staphylococcal infections including the presence of foreign bodies, or any patient prior to release from a hospital. The use of lysostaphin intranasals as a pre-release treatment will serve to inhibit community spread of hospital-acquired staphylococcal strains. Among non-human patients, those at risk include zoo animals, herd animals, and animals maintained in close quarters, such as swine, kenneled and stabled animals.

The lysostaphin intranasals of the invention provide several benefits not afforded by previous anti-staphylococcal treatments. First, the viscosity and mucoadhesive properties of a lysostaphin intranasal leads to longer retention time in the mammalian nares. Longer retention allows for greater exposure to staphylococci in the nares, thus increasing effectiveness and requiring fewer applications than alternate treatments. Second, application of lysostaphin intranasals to the mammalian nares does not lead to the emergence of lysostaphin resistant staphylococci. In contrast to previous studies that have reported the development of lysostaphin resistant bacteria when lysostaphin was administered systemically (17), the inventors of the instant invention have made the surprising discovery that, in the mammalian nares, lysostaphin-resistant staphylococci fail to emerge. Thus, lysostaphin intranasals are particularly useful with bacteria where antibiotic resistance is a problem.

Finally, lysostaphin intranasals that comprise recombinant lysostaphin have a greater specific activity, i.e., amount of activity per volume of formulation. Lysostaphin is naturally produced by bacteria as a pro-enzyme that is later proteolytically processed to produce the mature protein. When lysostaphin is isolated from bacteria, both the active form and the less active pro-enzyme form are present in the resulting preparation. The pro-enzyme form is approximately four-fold less active than the mature, active form (67). Active forms of naturally produced lysostaphin include a heterologous mix of polypeptides. This heterology is due to proteolytic processing of the pro-enzyme of lysostaphin. This proteolytic processing occurs at a number of different sites near the N-terminus of full length lysostaphin and leads to a heterologous mix of final active lysostaphin molecules. This variability can differ among lysostaphin preparations derived from natural sources. The presence of less active forms of lysostaphin dilutes out the concentration of active lysostaphin in the preparation, thus decreasing the specific activity of a formulation containing naturally derived lysostaphin. In contrast, recombinant lysostaphin preparations contain a single fully active form of lysostaphin. In such a preparation, there is no less active form to dilute out the activity of the mature form of lysostaphin. Thus, lysostaphin intranasals that comprise recombinant lysostaphin have a higher specific activity than their naturally derived counterparts.

As noted above, nasal colonization is a primary reservoir for staphylococci, and a strong correlation has been demonstrated between staphylococcal nasal colonization and (i) subsequent staphylococcal infections in those colonized; (ii) the potential to spread nasal colonization; and (iii) the potential for infection of other individuals near those colonized. This invention eradicates pre-existing staphylococcal nasal colonization, thereby reducing the chance of subsequent infection in the treated individuals or spread of S. aureus nasal colonization to others. Moreover, the eradication of pre-existing staphylococcal nasal colonization reduces the overall frequency of staphylococcal infections in the general population by eliminating a primary reservoir. Global reduction of staphylococcal infections in a community is especially important given the emergence of antibiotic-resistant staphylococcal strains, such as MRSA. Reducing the number of new staphylococcal infections in turn reduces the rate at which new resistant strains appear in the general population.

Among the staphylococcal organisms to be targeted by the invention is S. aureus. These lysostaphin intranasal compositions can be used to reduce or eradicate S. aureus nasal reservoirs in a general population, thus reducing subsequent staphylococcal infections and the spread of drug resistant S. aureus as discussed above. Administration to all or a portion of a patient population, for example, hospitalized patients, healthcare providers, pigs, cattle, sheep, goats, or other herded animals, may increase the overall health of the population.

It should be recognized that lysostaphin intranasals may also be used in combination with other formulations. These formulations may contain, for example, monoclonal antibodies that recognize staphylococcal antigens.

### BRIEF DESCRIPTION OF THE DRAWINGS

FIG. 1 shows that lysostaphin, when delivered in a cream formulation, remains in the nares for longer periods of time than does lysostaphin delivered in a PBS solution.

FIG. 2 shows that nasal cream is just as effective at retaining an antibacterial agent in the nares as polystyrene sulfonate (PSSA) or PSSA mixed with cream.

FIGS. 3A and 3B show that both nisin cream and lysostaphin cream have good anti-staphylococcal activity in vitro.

### DETAILED DESCRIPTION OF THE INVENTION

One aspect of the invention is directed to a cream formulation comprising lysostaphin useful for eradicating staphylococcal nasal colonization. The lysostaphin cream may also contain additional ingredients that increase its viscosity and make it mucoadhesive, thereby enhancing the retention time of lysostaphin in the nares. These ingredients include, for example, a cream base, consistency regulators, emulsifiers, and stabilizers. The cream base is responsible for most of the viscosity of the cream formulation. Consistency regulators serve to harden the cream formulation and also may affect viscosity. Emulsifiers and stabilizers contribute mostly to the stability of the cream formulation, but may also affect viscosity. The lysostaphin intranasals of the invention may be introduced into the mammalian nares by several methods that include applying the lysostaphin intranasal with a sterile swab, squeezing a tube of lysostaphin intranasal into the nares followed by massaging the nose, and squeezing an amount onto the finger of a patient for application to the nares or anterior nares, or via any type of delivery device.

In another aspect of the invention, the lysostaphin intranasal may be in a viscous liquid form or spray form and include various nasal delivery vehicles and/or carriers. Such vehicles may enhance the retention time of lysostaphin in the mammalian nares. These carriers include, for example, polyphosphoesters, polyethylene glycol, and high molecular weight poly (lactic acid), microsphere encapsulations, hydroxypropyl cellulose, chitosan, and polystyrene sulfanate. Such liquid formulations may be administered by aerosol or spraying into the nares, or introducing droplets into the nares. In addition, both cream and liquid intranasals may also include other antibacterials such as bacitracin, beta-lactams, polysporins, glycopeptides, lantibiotics like nisin or subtilin, and any other antibiotic with anti-staphylococcal action that can be applied intranasally.

Another aspect of the invention is directed to a method of administering the lysostaphin intranasals of the invention to the mammalian nares to eradicate, alleviate, or block colonization of the nares by staphylococci. The lysostaphin intranasals may be administered either singularly or in combination with other antibacterial agents such as β-lactams, antibodies, and lantibiotics like nisin or subtilin, and other antibiotics like bacitracin or neomycin or other anti-staphylococcal enzymes like mutanolysin, lysozyme or cellozyl muramidase.

The term “lysostaphin,” as used herein, encompasses any enzyme or anti-staphylococcal agent having proteolytic activity, in vitro and in vivo, against glycine-containing bridges in the cell wall peptidoglycan of staphylococci. The compositions of the invention are therefore applicable against any bacteria susceptible to attack by lysostaphin activity. Lysostaphins within the scope of the invention encompass: wild-type lysostaphin and related proteins or anti-staphylococcal agents, lysostaphin mutants, variants, fully synthetic and partially synthetic lysostaphins, human or animal lysostaphins, and recombinantly expressed lysostaphin proteins. Lysostaphin variants may be generated by post-translational processing of the protein (either by enzymes present in a producer strain or by means of enzymes or reagents introduced at any stage of the process) or by mutation of the structural gene. Mutations may include site-deletion, insertion, point mutations, domain removal and replacement mutations. Lysostaphin includes, for example, lysostaphin purified from S. simulans, Ambicin L (recombinant lysostaphin produced in Bacillus sphaericus and available from Nutrition 21, formerly AMBI), and mature lysostaphin purified from a Lactococcus lactis expression system or an E. coli expression system, and truncated lysostaphin as set forth in copending application, Truncated Lysostaphin Molecule With Enhanced Staphylolytic Activity, filed herewith, and specifically incorporated by reference.

The term “lysostaphin cream,” as used herein, means a cream-based formulation comprising lysostaphin as an active ingredient. A lysostaphin cream may be comprised of an amount of lysostaphin anywhere from 0.125% to 10% or more, recognizing that optimal dosages may differ by only 0.05%. Thus, in representative embodiments, lysostaphin may be present in at least any of the following concentrations: 0.125%, 0.25%, 0.5%, 0.75%, 1.0%, 1.25%, 1.50%, 1.75%, 2.0%, 2.25%, 2.50%, 2.75%, 3.0%, 3.25%, 3.50%, 3.75%, 4.0%, 4.25%, 4.50%, 4.75%, 5.0%, 5.25%, 5.50%, 5.75%, 6.0%, 6.25%, 6.50%, 6.75%, 7.0%, 7.25%, 7.50%, 7.75%, 8.0%, 8.25%, 8.50%, 8.75%, 9.0%, 9.25%, 9.50%, 9.75%, or 10% lysostaphin.

As discussed above, the cream formulation, to which lysostaphin is added, may be comprised of a cream base, consistency regulators, emulsifiers, and stabilizers. Components of a cream base may include, for example, petrolatum and SOFTISAN 649 (Sasol, Inc.) (Bis-Diglyceryl Polyacyladipate-2). Consistency regulators may include, for example, paraffin and beeswax. Emulsifiers and stabilizers may include, for example, MIGLYOL 812 (Sasol, Inc.) (Caprylic/Capric Triglyceride), zinc stearate, and aluminum stearate. In one embodiment, the cream formulation is comprised of 15%-50% MIGLYOL 812, 15%-50% SOFTISAN 649, 15%-50% White Petrolatum, 0%-10% Paraffin, 0%-10% Beeswax, and 0%-5% Aluminum Stearate. In another embodiment, the cream formulation is 36% MIGLYOL 812, 24.20/% SOFTISAN 649, 27.5% White Petrolatum, 3.4% Paraffin, 3.4% Beeswax, and 0.5% Aluminum Stearate. In another embodiment, Zinc Stearate may be substituted or partially substituted for Aluminum Stearate (collectively “metal stearate”).

In yet another embodiment, the cream formulation is 41% MIGLYOL 812, 24.2% SOFTISAN 649, 27.5% White Petrolatum, 3.4% Paraffin, 3.4% Beeswax, and 0.5% Zinc Stearate. When adding lysostaphin to the cream formulation, the lysostaphin replaces part of the MIGLYCOL 812 content. For example, if 5% of the cream formulation were comprised of a lysostaphin solution, then MIGLYOL 812 would comprise 36% of the formulation.

The term “lysostaphin liquid,” as used herein, means a viscous liquid-based formulation comprising lysostaphin as an active ingredient and a polymer. A lysostaphin liquid may be comprised of an amount of lysostaphin anywhere from 0.125 to 10% or more, recognizing full optimal dosages may differ by only 0.05%. Thus, lysostaphin may be present in at least any of the following concentrations: 0.125%, 0.5%, 0.75%, 1.0%, 1.25%, 1.50%, 1.75%, 2.0%, 2.25%, 2.50%, 2.75%, 3.0%, 3.25%, 3.50%, 3.75%, 4.0%, 4.25%, 4.50%, 4.75%, 5.0%, 5.25%, 5.50%, 5.75%, 6.0%, 6.25%, 6.50, 6.75%, 7.0%, 7.25%, 7.50%, 7.75%, 8.0%, 8.25%, 8.50%, 8.75%, 9.0%, 9.25%, 9.50%, 9.75%, 10%, or more lysostaphin. The liquid formulation, to which lysostaphin is added, may be comprised of at least one of hydroxypropyl cellulose, chitosan and polystyrene sulfonate. The term “lysostaphin intranasal” means a viscous formulation comprising lysostaphin and includes lysostaphin creams and lysostaphin liquids.

The term “retention time,” as used herein, means the length of time between the initial introduction of a lysostaphin intranasal to the mammalian nares and the absence of lysostaphin or antibacterial lysostaphin activity in the mammalian nares.

A lysostaphin intranasal is said to “alleviate” staphylococcal colonization if it is able to decrease 1) the number of colonies in the nares of a mammal, or 2) the frequency of positive nasal cultures for the presence of S. aureus; when the lysostaphin intranasal is administered before, concurrently with, or after exposure to staphylococci, whether that exposure results from the intentional instillation of staphylococci or from general exposure. For instance, a lysostaphin intranasal is considered to alleviate colonization if the number of bacterial colonies that can be grown from a sample of nasal tissue, or nasal swab, is decreased after administering the lysostaphin intranasal. A lysostaphin intranasal alleviates colonization, as in the nasal colonization assays described herein, when it decreases the number of colonies by at least 30%, at least 40%, at least 50%, at least 60%, at least 75%, at least 80%, at least 90%, or by 100%. One hundred percent alleviation would be “eradication.”

A lysostaphin intranasal is said to “block” staphylococcal colonization if it is able to prevent the nasal colonization of a mammal when the lysostaphin intranasal is administered prior to, or concurrently with, exposure to staphylococci, whether by intentional instillation or otherwise into the nares. A lysostaphin intranasal blocks colonization as in the nasal colonization assay described herein, if no staphylococcal colonies can be grown from a sample of nasal tissue taken from a mammal treated with the lysostaphin intranasal of the invention for an extended period, such as 12 hours or longer or 24 hours or longer compared to control mammals.

In a clinical setting, the presence or absence of nasal staphylococcal colonization in a human patient is determined by culturing nasal swabs on an appropriate bacterial medium often after an overnight enrichment step in a broth culture. These cultures are scored for the presence or absence of staphylococcal colonies. In this type of qualitative assay system, it may be difficult to distinguish between blocking and alleviation of staphylococcal colonization. Once blocking or alleviation have occurred, the patient may be recolonized from an external source. Thus, for the purposes of qualitative assays, such as nasal swabs, a lysostaphin intranasal “blocks” colonization if a human patient at risk for nasal colonization, who at the time of treatment tests negative for nasal colonization, remains negative for nasal colonization for an extended period, such as 12 hours or longer or 24 hours or longer. A lysostaphin intranasal “alleviates” staphylococcal nasal colonization in a human patient if it causes a discernable decrease in the frequency of positive cultures or recoverable bacteria taken from a human patient who is already positive for staphylococci before the lysostaphin intranasal of the invention is administered. A lysostaphin intranasal “eradicates” nasal colonization if after application of material there are no positive cultures taken from a human patient who had positive cultures prior to the application.

Another aspect of the invention is directed to a method of eradicating, alleviating, or blocking secondary staphylococcal infections in patients with respiratory viral infections, transplant patients, HIV infected patients, burn patients, patients with intravascular devices or foreign bodies, convalescing patients, and other such patients that are subject to secondary infection by administering the lysostaphin intranasals noted above in order to eliminate a primary reservoir for subsequent staphylococcal infection.

The method of the invention also includes the eradication, alleviation, or blocking of nasal colonization by any clinical isolate of staphylococci, including any of the various capsule types, as well as strains that are resistant to methicillin, vancomycin, mupirocin and other antibiotics, by such administrations. Furthermore, the invention has the added benefit of inhibiting the spread of antibiotic-resistant strains of staphylococci to the community by eradicating nasal colonization in people released from health care settings, a primary reservoir for antibiotic-resistant strains of staphylococci.

Because a goal of the invention is to reduce the frequency of staphylococcal infections, the instillation of an effective amount of the lysostaphin intranasal of the invention includes that sufficient to demonstrate a medically meaningful, discernable, or statistically significant of decrease in the likelihood of staphylococcal infection, for example systemic infection, or infections at the site of trauma or surgery. Such demonstrations may encompass, for example, animal studies or clinical trials of patients at risk, including health care workers, newborns and premature infants, persons undergoing inpatient or outpatient surgery, burn victims, patients receiving indwelling catheters, stents, joint replacements and the like, geriatric patients, and those with genetically, chemically or virally suppressed immune systems.

As used herein, “treatment” encompasses the administration of an effective amount of a compositions of the invention to the nares of a patient in one or more doses. An effective amount is that sufficient to result in a medically meaningful, discernable, or statistically significant reduction, amelioration, alleviation, or eradication of existing colonization by S. aureus or other staphylococci, as well as blocking or prophylaxis against future colonization. Treatment of a patient thus results in a “therapeutically beneficial outcome,” hereby defined as any of: 1) no nasal colonization by staphylococci for at least 12 hours after a final instillation of the composition, 2) a medically meaningful, discernable, or statistically significant decrease in the number of staphylococcal colonies in the nares within 4 hours, within 12 hours, or within 24 hours after final instillation of the composition, 3) a decrease in the frequency of positive cultures taken from the nares within 4 hours, within 12 hours, within 24 hours after final instillation of the composition; 4) continued activity of the lysostaphin in the nares for at least 12 hours, at least 24 hours, at least 48 hours after final instillation of the composition, 6) eradication, alleviation, or blockage of colonization of the mammalian nares by staphylococci by a single dose of the composition, by two doses, by three doses, by four doses, by five doses, by six doses, by seven doses, by eight doses, by nine doses, by ten doses; 7) any medically meaningful, discernable, or statistically significant blocking or prophylaxis against future staphylococcal colonization; or 8) any medically meaningful, discernable, or statistically significant reduction in the likelihood of staphylococcal infection in the treated patient including nosocomial staphylococcal infection.

Treatment thus encompasses a medically meaningful, discernable, or statistically significant reduction in the number of staphylococci in the nares of a colonized patient as well as a reduction in likelihood of future colonization or staphylococcal infection. As used herein, “colonized” refers to the subclinical presence of staphylococcal bacteria in the nares of a patient, whereas “infected” refers to clinical infection in any body site. A “medically meaningful” treatment encompasses any treatment that improves the condition of a patient; improves the prognosis for a patient; reduces morbidity or mortality of a patient; reduces the likelihood of future colonization or infection; or reduces the incidence of morbidity or rates of mortality from the bacterial infections addressed herein, among a population of patients. The specific determination or identification of a “statistically significant” result will depend on the exact statistical test used. One of ordinary skill in the art can readily recognize a statistically significant result in the context of any statistical test employed, as determined by the parameters of the test itself. Examples of these well-known statistical tests include, but are not limited to, X² Test (Chi-Squared Test), Student's t Test, F Test, M test, Fisher Exact Text, Binomial Exact Test, Poisson Exact Test, one way or two way repeated measures analysis of variance, and calculation of correlation efficient (Pearson and Spearman).

The lysostaphin intranasal compositions of the invention are administered into the nares of humans. Intranasal administration of compounds containing lysostaphin has been reported in the literature as effective in treating nasal carriers of staphylococci, as demonstrated in three independent studies. First, in a study by Martin and White, these authors tested the use of a 0.5% lysostaphin saline spray on adults who were colonized with S. aureus (42). Each participant in the study self-applied the spray to each nostril, three times per day for seven to twelve days. Martin and White noted a decrease in the number of nasal cultures positive for S. aureus from 100% to 20% by the end of the treatment schedule. In addition, the number of S. aureus colonies isolated from subjects who remained carriers also decreased.

Second, in a subsequent study, Harris et al. tested the use of 0.5% lysostaphin in saline on infants and children (28). Patients received a lysostaphin spray 4 times per day for seven to fourteen days. S. aureus colonization was eradicated in ten out of ten subjects. Seventy percent of the patients remained colony free for sixteen days or more. Harris et al. did note immune sensitivity to lysostaphin in one of the test subjects. As these authors indicated, at the time, lysostaphin preparations were contaminated with other proteins, and other studies indicated that lysostaphin was capable of inducing antibody formation and anaphylactic shock in animals.

Finally, in a study by Quickel et al., a 0.5% lysostaphin in saline spray was used to treat several adult patients nasally colonized with S. aureus (57). Patients were divided into three treatment groups. The first group received lysostaphin spray treatment three times per day for five days. The second treatment group received Neosporin ointment. The third treatment group received no therapy. After completion of the treatment schedules, 40% of the lysostaphin-treated patients still carried S. aureus and 60% of the patients were carriers by day 5 post-treatment. As with the Harris et al. study, Quickel et al. also noted signs of an immune response in some patients and suggested that more testing was necessary to prove the safety of lysostaphin for use in humans.

Taken together, in all three of these studies, numerous doses of naturally-derived lysostaphin in saline were used on the test subjects. Some test subjects remained colonized despite the aggressive dosing schedule used in these studies. Other subjects did show eradication of staphylococcal colonization in the nose, but again this was after treatment with several doses per day over several days. Even if such a dosing schedule did cause eradication in all test subjects, the likelihood that a patient would or could follow such a lengthy and complex dosing schedule to its completion is questionable. The simpler the dosing regime, the more likely a patient or health care provider is to apply a treatment successfully. Also, as noted above, in some of the studies, immune reactions to lysostaphin were noted in some of the test subjects, which may have been due to the repeated exposure to lysostaphin. Given the shortcomings of the previous studies, the inventors sought to create a lysostaphin intranasal that in very few doses or even one dose can quickly eradicate or alleviate nasal colonization by staphylococci.

The resulting lysostaphin intranasal of the invention improves over these studies in two ways. First, the lysostaphin used in the previous studies was natural lysostaphin purified from S. simulans. Lysostaphin is naturally produced by bacteria as a pro-enzyme that is cleaved in a series of steps to produce the full length, fully active form of lysostaphin. When lysostaphin is isolated from bacteria, both the active form and the less active pro-enzyme are present in the resulting preparation (67). The presence of less active forms of lysostaphin dilutes out the concentration of fully active lysostaphin in the preparation, thus decreasing the specific activity of a formulation containing naturally derived lysostaphin. In contrast, in some embodiments, the present invention uses recombinant lysostaphin preparations, which contain only a single fully active form of lysostaphin. In such a preparation, there are no less active forms to dilute out the activity of the mature form of lysostaphin. Thus, the specific activity (amount of activity per volume of preparation) of a lysostaphin intranasal made with recombinant lysostaphin is higher than the specific activity of a lysostaphin intranasal made from a natural source of lysostaphin, and the resulting recombinant lysostaphin preparation is free from contaminating cell products from S. simulans.

Second, the inventors have demonstrated that administration of lysostaphin in a cream formulation improves retention of lysostaphin in the nares and they believe that a viscous liquid formulation would also improve retention time in the nares. An improved retention time can improve the effectiveness of any lysostaphin intranasal, whether made with naturally-derived lysostaphin or recombinant lysostaphin.

As discussed above, in at least one embodiment of the invention, the inventors combined three benefits: (i) an improved retention time over saline; (ii) the use of a recombinant lysostaphin that has a higher specific activity than naturally-derived lysostaphin; and (iii) the use of a homogenous preparation of lysostaphin. The presence of lysostaphin molecules of differing N-terminal amino acids in a heterogenous preparation of lysostaphin makes it more difficult to analyze the “purified” product for contaminants. Thus, with homogenous lysostaphin preparations, detection of contaminants is more readily achieved.

As a result of these improvements, the inventors demonstrate below that a single dose of lysostaphin intranasal totally eradicated S. aureus nasal colonization in a cotton rat animal model. This eradication has been demonstrated to last at least a week, using the cotton rat animal model described below.

In another embodiment, the lysostaphin intranasal of the invention may be administered in conjunction with other anti-staphylococcal drugs including antibiotics like mupirocin and bacitracin; anti-staphylococcal agents like lysozyme, mutanolysin, and cellozyl muramidase; anti-staphylococcal antibodies; anti-bacterial peptides like defensins; and lantibiotics, or any other lanthione-containing molecule, such as nisin or subtilin.

In view of the disclosure provided, the administration of the lysostaphin intranasal of the invention is within the know-how and experience of one of skill in the art. In particular, the amount of lysostaphin intranasal required, combinations with appropriate carriers, the dosage schedule and amount may be varied within a wide range based on standard knowledge in the field without departing from the claimed invention. In one embodiment, the lysostaphin cream may be administered once, twice, or three times a day for between 1 and 5 days. In another embodiment, the lysostaphin cream may be administered once per day at 0.5% to 2.0% per dose. These doses are known to be effective with an initial inoculum of 10⁹ S. aureus bacteria, an amount known to ensure 100% colonization in an animal model (33). An initial dose of 10⁹ S. aureus generally leads to nasal colonization of 10 to 10 CFUs per animal nose five days post-instillation of bacteria. This level of intranasal colonization can last for at least one month post-instillation. Such a lysostaphin dosing regimen would be effective on very young patients, very old patients, convalescing patients, pregnant mothers, patients either admitted to the hospital for surgical procedures, patients suffering from various conditions that predispose them to staphylococcal colonization, or prior to their release from hospitals. A patient can be any human or non-human mammal in need of prophylaxis or other treatment. Representative patients intended for nasal instillation are any mammal subject to S. aureus or other staphylococcal infection or carriage, including humans and non-human animals such as mice, rats, rabbits, dogs, cats, pigs, sheep, goats, horses, primates, ruminants including beef and milk cattle, buffalo, camels, as well as fur-bearing animals, herd animals, laboratory, zoo, and farm animals, kenneled and stabled animals, domestic pets, and veterinary animals.

The present invention is further illustrated by the following examples that teach those of ordinary skill in the art how to practice the invention. The following examples are merely illustrative of the invention and disclose various beneficial properties of certain embodiments of the invention. The following examples should not be construed as limiting the invention as claimed.

### EXAMPLES

### Example 1

### Lysostaphin in Phosphate Buffered Saline

As discussed above, the inventors sought to create a lysostaphin intranasal that in very few doses or even one dose can quickly eradicate or alleviate nasal colonization by staphylococci. The studies by Martin and White, Harris, and Quickel used naturally-derived lysostaphin, which contains both the less active pro form of lysostaphin and the proteolytically processed fully active form. In an initial attempt to improve treatment of nasal colonization by staphylococci, the inventors used recombinant lysostaphin in saline to treat nasal colonization in cotton rats. By using recombinant lysostaphin, which lacks the less active pro-form of lysostaphin and contains only fully active lysostaphin, the inventors were able to increase specific activity of the lysostaphin intranasal over intranasal formulations using naturally-derived lysostaphin.

The efficacy of lysostaphin in phosphate buffered saline (PBS) was tested in a cotton rat animal model for nasal S. aureus colonization. Four to six week old Sigmadon hispidis cotton rats were given sterile water containing nafcillin (1 g/l) ad libitum (as much as the animal desired) ?4 hours prior to bacterial instillation. Although administration of nafcillin decreases the competition for growth by endogenous bacteria in the nose, thereby enhancing the ability of the experimental MRSA strain to establish colonization in the nares, nafcillin is not absolutely necessary to establish MRSA colonization. At the same time, a Columbia agar plate containing 2% NaCl (CSA) was inoculated with S. aureus strain MBT 5040 from a frozen stock. MBT 5040 is a clinical MRSA strain isolated from tissue and has one of the highest minimal inhibitory concentrations (MIC) for lysostaphin in the inventors' collection. This strain came from the Walter Reed Army Medical Center (WRAMC). The methicillin MIC for MBT 5040 is >36 μg/ml. It should be noted that the MIC of lysostaphin for MBT 5040 is 0.064 μg/ml which is one of the higher MICs tested thus making MBT 5040 is a good representative strain of S. aureus for use in this model. The MIC of a drug for a particular bacterial strain is the minimum concentration of the drug that inhibits normal growth of that particular bacterial strain. Growth on CSA plates encourages capsule formation around the bacteria, which in turn yields more efficient colonization of the nares.

On the day of instillation, S. aureus MBT 5040 was harvested from the CSA plate by scraping colonies into sterile. PBS (1 ml/animal to be instilled) until the percent transmittance of the sample was approximately 10% at 650 nM in a 10 mm path length. The bacteria were pelleted by centrifugation and then resuspended in 10 μl/animal of sterile PBS. Cotton rats were sedated with 200 μl of Ketamine (25 mg/kg), Rompun (2.5 mg/kg), and Acepromazine (2.5 mg/kg) delivered intramuscularly. Ten microliters, approximately 10⁹ S. aureus CFUs per animal, of MBT 5040 in PBS was instilled in the nares using a micropipette without touching the nares. Specifically, a drop of bacterial inoculum was placed on the nostril with a micropipettor, without touching the nose. The animal's regular process of respiration then inhaled the drop into the nares. After introduction of the MBT 5040 bacteria, the cotton rats were returned to normal water, without nafcillin. Unless otherwise indicated, this method was consistently used to instill S. aureus in the nares for all examples discussed below.

On day 4 and day 5 post-instillation, or day 5 only, animals were treated with lysostaphin in PBS or with PBS alone. In this example and in Examples 2-10, Ambicin L (Ambi, Inc.) was used as a source of recombinant lysostaphin. Animals were again anaesthetized and a 10 μl drop of PBS alone or lysostaphin in PBS (110 μg of lysostaphin/animal) was placed on the nostril for inhalation as described above. Two hours after the final treatment, the animals were sacrificed by CO₂ inhalation. The noses were wiped with a sterile 70% ethanol wipe before they were removed surgically, dissected, and vortexed well in 500 μl sterile PBS containing 0.5% Tween-20 to release colonizing bacteria. Fifty to 100 μl of PBS were plated on various types of agar plates to determine actual colonization and look for lysostaphin resistance. Specifically, lysostaphin resistance was monitored by determining the lysostaphin-sensitivity of colonies that grew on blood agar and tryptic soy agar (TSA) plus 7.5% NaCl without nafcillin or streptomycin. Because MBT 5040 S. aureus was nafcillin and streptomycin resistant, overall nasal colonization was measured as CFUs on TSA+7.5% NaCl, nafcillin, and/or streptomycin (10 mcg/ml and 500 mcg/ml respectively) plates. Microbiological tests were then used to determine which, if any, colonies on blood agar or TSA+7.5% NaCl were S. aureus. In cases where MRSA were treated with lysostaphin, supernatants were also planted on TSA+NaCl without antibiotics to allow growth of lysostaphin resistant colonies that may become methicillin sensitive. TSA plates supplemented with NaCl were incubated for 48 hours at 37° C. to allow S. aureus colonies to grow to a size that could be easily counted.

Any detected S. aureus after lysostaphin treatment were tested for lysostaphin resistance by microtiter dilution assay. Lysostaphin was resuspended in sterile PBS, aliquotted and stored at −80° C. A protein assay (e.g., Pierce BCA) was used to determine the actual protein concentration. Once thawed, an aliquot of lysostaphin was stored at 4° C. and used for no more than two weeks. Plates were prepared by making lysostaphin dilutions in cation-adjusted Mueller Hinton broth+2%NaCl and 0.1%BSA (CAMHB+). The CAMHB+2% NaCl was made first, autoclaved, and then sterile 30% BSA was added to equal 0.1% BSA concentration. BSA prevents nonspecific lysostaphin interaction with plastic. The final volume of CAMHB+ in each well was 50 μl. Dilutions were 1:2, prepared by mixing 50 μl of the previous dilution into 50 μl of fresh media. The final row on the plate was left with no lysostaphin added as a control for growth. A starting concentration of 1 μg/ml lysostaphin was used as the stock concentration. The stock concentration of lysostaphin was twice what was desired for the highest concentration in the assay to allow for an additional 1:2 dilution once the bacteria were added. Often 250 ng/ml, as the final concentration of lysostaphin in the first row, was an appropriate starting point for lysostaphin sensitive strains. Bacteria were grown in a non-selective media (tryptic soy broth) or on a non-selective agar (TSA+5% sheep's blood). Overnight cultures were diluted ˜1:1000, as determined empirically, to yield a final concentration of 5×10⁵/ml by measuring the optical density at 650 nm (OD₆₅₀). The final inoculum of bacteria per well was ˜5×10⁵ CFUs/ml. Each well of lysostaphin dilution series was inoculated with 50 μl (˜5×10⁴ CFUs) of the 1:1000 dilution in CAMHB+. The final volume per well was 100 μl. Plates were incubated 24 hrs with shaking at 37° C. The minimal inhibitory concentration (MIC) of lysostaphin, the lowest concentration of lysostaphin that prevents normal growth, was determined by reading the OD₆₅₀ on a microplate reader.

The inventors have discovered that most overnight S. aureus cultures in non-selective media contain a very small number of naturally occurring lysostaphin-resistant bacteria, and if by random chance a sufficient number of these resistant bacteria are inoculated in a well during MIC assay, regardless of the concentration of lysostaphin in that well, there may be an outgrowth of bacteria in that particular well. To verify that any samples positive for growth in the presence of a given concentration of lysostaphin were actually normal S. aureus as opposed to an outgrowth of small numbers of lysostaphin-resistant colonies selected for by the lysostaphin in that well, 25-50 μg/ml of extra lysostaphin in a 1-2 μl volume were added to each well that exhibited growth. Plates were incubated at room temperature for 6 to 18 hours. The OD₆₅₀ was determined. If the OD₆₅₀ dropped substantially following the addition of excess lysostaphin, then the bacteria in that well were considered normal such that the concentration of lysostaphin originally in that particular well was below the MIC for that strain. If the OD₆₅₀ stayed the same or increased, then the bacteria in that sample were considered lysostaphin-resistant outgrowth and not normal growth below the MIC of lysostaphin for that strain. Resistance outgrowth may be seen at different concentrations. Truly resistant isolates display growth and subsequent lysostaphin resistance in all concentrations of lysostaphin tested and in control wells receiving no initial lysostaphin. It would be apparent to one of ordinary skill in the art that under the confines of this assay, some staphylococci may be so resistant that a MIC may not be detectable.

As shown in Tables 1 a and 1 b below, recombinant lysostaphin in PBS failed to eradicate nasal colonization in three out of four animals when two treatments were given. Thus, recombinant lysostaphin in PBS, when used in two treatments on colonized animals in Table 1 a and one treatment in Table 1 b, was not very effective in eradicating nasal colonization and demonstrated a marginal ability to alleviate colonization. No lysostaphin resistant S. aureus were isolated.

TABLE 1a
Number of CFUs recovered in 100 μl of
PBS + Tween-20 - Experiment 1

| | | Lysostaphin in |
|---|---|---|
| Animal | PBS alone | PBS |
| 1 | 857 | 690 |
| 2 | 1369 | >1200 |
| 3 | 1436 | 115 |
| 4 | 862 | 0 |
| Average number of | 1131 | 501 |
| CFU | | |

TABLE 1b
Number of CFUs recovered in 100 μl of
PBS + Tween-20 - Experiment 2

| | | Lysostaphin in |
|---|---|---|
| Animal | PBS alone | PBS |
| 1 | 610 | 536 |
| 2 | 1223 | 8 |
| 3 | 696 | 4 |
| 4 | 634 | 0 |
| 5 | 9 | 0 |
| Average number of | 634 | 183 |
| CFU | | |

### Example 2

### Lysostaphin in a Cream Formulation

In light of the above observations, the inventors further improved on the lysostaphin intranasal of Example 1 by creating a more viscous formulation that would allow longer retention of lysostaphin in the nose. To this end, the inventors used a lysostaphin cream to treat S. aureus nasal colonization in cotton rats.

The efficacy of lysostaphin in a cream formulation was also tested in the cotton rat model. The cream formulation consisted of MIGLYOL 812 (Caprylic/Capric Triglyceride) (41%), SOFTISAN 649 (Bis-Diglyceryl Polyacyladipate-2) (24.2%), white petrolatum (27.5%), paraffin (3.4%), beeswax (3.4%), and aluminum stearate (0.5%). For production of a lysostaphin intranasal, recombinant lysostaphin, or Ambicin L (Ambi, Inc.), was dissolved in sterile PBS to a concentration of 100 mg/ml. This lysostaphin solution was then mixed with the above cream formulation to the desired final concentration. The volume taken up by the addition of lysostaphin replaced part of the MIGLYOL 812 content in the resulting lysostaphin cream. Thus, the final formulation of lysostaphin cream used was MIGLYOL 812 (36%), Softisan 649 (24.2%), white petrolatum (27.5%), paraffin (3.4%), beeswax (3.4%), aluminum stearate (0.5%) and 5% of 100 mg/ml aqueous lysostaphin, yielding a final lysostaphin concentration of 0.5% or 5 mg/ml.

Ten cotton rats were nasally instilled with MBT 5040 S. aureus. On day 4 and day 5 post-instillation, the 5 animals were treated with a 0.5% lysostaphin cream and 5 animals were treated with a cream without lysostaphin added. Cream formulations were delivered into the nares of anaesthetized cotton rats by syringe through an Angiocath™ 22 GA flexible catheter (Becton Dickinson). The catheter was inserted approximately 3 mm into each of the nares and then drawn back as the cream was delivered. The noses of the cotton rats were massaged well following cream delivery to ensure good dispersal of cream. The dose of cream delivered to the nares was approximately 30-50 μl. Due to the cream's highly viscous nature, it was difficult to precisely gauge to volume of creams delivered to the nares. Unless otherwise indicated, this method of cream delivery into the nares was used throughout the examples below.

As shown in Table 2a, nasal delivery of lysostaphin in a cream formulation resulted in greatly improved rates of eradication of colonization when compared to treatment with PBS solutions. S. aureus colonization was not eradicated in any of the negative control animals treated with cream lacking lysostaphin. In contrast, nasal colonization in 4 out of 5 animals treated with lysostaphin cream was completely eradicated. In the fifth cotton rat, nasal colonization was alleviated to a greater extent than that observed with lysostaphin in PBS as discussed above. The colonies that remained in the animal treated with lysostaphin cream were not lysostaphin resistant.

TABLE 2a
Number of CFUs recovered in 100 μl of PBS + Tween-20

| | | 0.5% Lysostaphin |
|---|---|---|
| Animal | Negative Control Cream | Cream |
| 1 | 280 | 7 |
| 2 | 1213 | 0 |
| 3 | 148 | 0 |
| 4 | 402 | 0 |
| 5 | -¹ | 0 |
| Average number of | 510 | n/a |
| CFU | | |

¹This animal perished before completion of the experiment due to complications not related to staphylococcal colonization.

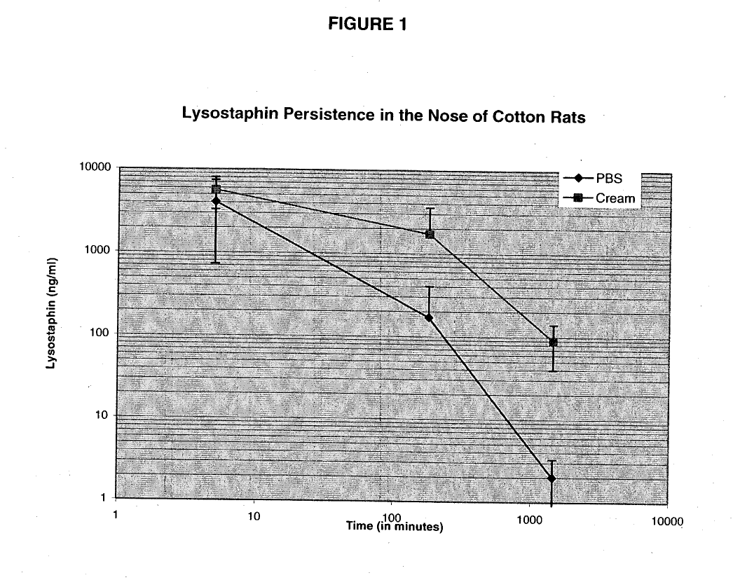

To address a possible mechanism to account for the greater efficacy of lysostaphin in a cream formulation at eradicating nasal colonization, the nasal retention time was measured in rats treated with either lysostaphin in PBS or lysostaphin in a cream. Twelve animals were given 0.5% lysostaphin in PBS and another 12 were given 0.5% lysostaphin cream. At 5 minutes, 3 hours, and 24 hours post-instillation, 4 animals in each group were sacrificed. Lysostaphin concentrations in the nose were then determined by ELISA. As shown in FIG. 1, lysostaphin when delivered in a cream formulation remains in the nares for longer periods of time than does lysostaphin delivered in a PBS solution.

Further, when 0.5% lysostaphin cream was administered to the nares in the absence of instilled staphylococcal colonization and then the nares were assayed for the presence of bactericidal activity, lysostaphin retained its bactericidal activity in the nares for at least 24 hours post-administration. As shown in Table 2b, the anti-staphylococcal activity of lysostaphin formulated in a petrolatum based-cream was retained intranasally for at least 24 hrs post instillation. When cotton rats were instilled with 0.5% lysostaphin in cream and the noses were surgically removed 4 or 24 hrs post instillation, there was sufficient residual lysostaphin activity to eliminate 10³ S. aureus ex vivo. The 0.125% lysostaphin cream reduced the S. aureus by 87% 4 hrs post instillation, while placebo cream resulted in only a small reduction, 4 or 24 hrs post instillation, in the number of recovered S. aureus added to the excised noses. Thus, the longer retention of lysostaphin in the nares, the site of S. aureus colonization, allowed longer exposure of the bacteria to lysostaphin. Longer exposure would in turn improved the ability of the lysostaphin cream to eradicate nasal colonization.

TABLE 2b

| | | Percent Reduction |
|---|---|---|
| Treatment Group | Treatment Time¹ | in CFUs² |
| Control (no cream) | — | 0 |
| Placebo cream | 4 hours | 30 |
| Placebo cream | 24 hours | 20 |
| 0.5% Lysostaphin (˜150 μg) | 4 hours | 100 |
| 0.5% Lysostaphin (˜150 μg) | 24 hours | 100 |
| 0.125% Lysostaphin (˜37.5 μg) | 4 hours | 87 |
| 5% Nisin (˜1500 μg) | 2 hours | 35 |

¹Time post-instillation of cream when nose was harvested and incubated with 10³ exogenous S. aureus.
²Determined in relationship to the control sample. Mean of three samples.

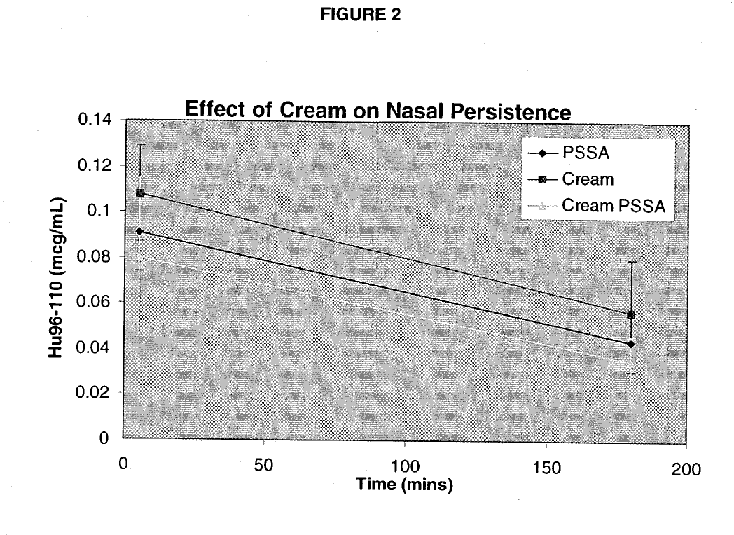

In addition, the cream formulation of the invention retains an antibacterial agent in the nares just as efficiently as other forms of delivery, such as micro-encapsulation. FIG. 2 demonstrates that, when compared to polystyrene sulfonate (PSSA) or PSSA mixed with cream, the cream formulation alone leads to comparable retention times for an antibacterial agent such as an anti-staphylococcal monoclonal antibody. MAb was mixed with 0.5% PSSA solution (in PBS) to final concentration of 5 mcg/mL. In one group, this solution was applied directly to the nose. In another, it was first mixed with the cream and then applied to the nose.

Since sufficient anti-staphylococcal activity of lysostaphin formulated in cream to eliminate 10³ S. aureus was retained for at least 24 hrs post instillation (Table 2b) and 10 μg of lysostaphin (˜7% of the original dose of lysostaphin instilled intranasally) will eliminate 10⁵ S. aureus within 20 min (Table 2c), it was necessary to identify a neutralizing substance for lysostaphin which would effectively eliminate lysostaphin activity in excised noses but not impair the viability or growth of residual S. aureus in the nose. A number of potential neutralizers were tested including, 0.5M EDTA, pH 3.6 buffer, 10 mg/ml trypsin, various protease inhibitors and excess quantities of heat killed S. aureus; none of these significantly inhibited lysostaphin activity in vitro (data not shown).

As shown in Table 2c, 10 mg/ml of Proteinase K, however, rapidly neutralizes lysostaphin in buffer but does not affect the viability of S. aureus. Lysostaphin (10 mcg) was added to some samples of 10⁵ S. aureus in PBS in the presence or absence of 10 mg/ml Proteinase K. The samples were incubated for 20 minutes at room temperatures and then 100 microliter aliquots were plated on blood agar.

TABLE 2c
Proteinase K Neutralizes Lysostaphin Activity in Vitro

| Sample | CFUs Recovered |
|---|---|
| Control (buffer only) | 10⁵ |
| +Proteinase K | 10⁵ |
| +Lysostaphin | 0 |
| +Lysostaphin and Proteinase K | 10⁵ |

Lysostaphin was administered in a GMP cream as described in (Example 11).

In follow up experiment, 0.5% lysostaphin GMP cream was instilled in naive cotton rat noses in an experiment similar to those in Table 2b. Three hours post instillation the animals were sacrificed and the excised noses placed in 500 μl of PBS/Tween+/−10 mg/ml Proteinase K. S. aureus strain MBT 5040 (10⁵ CFUs) was immediately added to the excised noses which were vortexed and incubated for 30 minutes. A volume of the supernatant was plated for enumeration. As shown in Table 2d, the residual lysostaphin in sample noses at 3 hours when placed in PBS/Tween without Proteinase K greatly reduced the viable S. aureus in the samples. The presence of 10 mg/ml Proteinase K neutralized all residual lysostaphin in the noses at 3 hours, i.e., there was no reduction in the number of S. aureus recovered from samples when the noses were placed in PBS/Tween+10 mg/ml Proteinase K.

TABLE 2d
Proteinase K Neutralizes Residual Lysostaphin in the
Nose 3 hours after Instillation of GMP 0.5% Lysostaphin Cream

| | Average CFUs |
|---|---|
| Sample | Recovered⁽²⁾ |
| Control | 10^{5 (3)} |
| +Excised Nose⁽¹⁾ | 52 |
| +Excised Nose and Proteinase K⁽¹⁾ | 10⁵ |

⁽¹⁾Noses were excised three hours post cream instillation.
⁽²⁾Results are the average of two samples.
⁽³⁾S. aureus MBT 5040 (10⁵ CFUs was added to the vortexed noses and incubated 30 minutes.

In order to confirm that the nasal clearance consistently seen with a single dose of 0.5% lysostaphin cream (Table 3b) occurs in the nose and not in buffer after excision of the nose by lysostaphin carryover, two groups of cotton rats nasally colonized with MBT 5040 were treated with a single application of 0.5% lysostaphin cream produced under GMP conditions. Four hours post-treatment, the animals were sacrificed and the noses excised. One group of noses was vortexed in PBS/Tween while the other group of noses was vortexed in PBS/Tween containing 10 mg/ml Proteinase K. Upon plating the supernatants from these excised noses, it was determined that all treated cotton rats were clear of S. aureus at 4 hours post treatment in the presence or absence of Proteinase K (5 of 5 and 4 of 4, respectively) (Table 2e). This finding demonstrated that lysostaphin eradication of S. aureus colonization in cotton rat noses occurred within 4 hours in the nose and not ex vivo by lysostaphin carryover following excision of the nose.

TABLE 2e
Lysostaphin Eradicates Nasal Colonization by
S. aureus MBT 5040 in Vivo Within 4 hours of
Treatment and Not ex Vivo due to Antibiotic Carryover

| | | Mean CFUs |
|---|---|---|
| | | Recovered per |
| Treatment of Cotton Rat Nose¹ | Nasal Colonization | Nose |
| Control (no treatment) | 4/4² | 1065 |
| Placed in Buffer | | |
| 0.5% Lysostaphin GMP cream | 0/4 | 0 |
| treated Placed | | |
| in Buffer | | |
| 0.5% Lysostaphin GMP cream | 0/5 | 0 |
| treated Placed | | |
| in Proteinase K¹ | | |

¹Treated noses were excised 4 hours post treatment.
²Animals colonized over animals tested.

### Example 3

### Titration of Lysostaphin Concentration in Nasal Creams

The concentration of lysostaphin was titered to determine the minimal concentration of lysostaphin in a cream formulation that would eradicate nasal colonization by S. aureus. Twenty cotton rats were instilled with MBT 5040 S. aureus. The animals were split into four treatment groups: negative control cream, 0.5% lysostaphin cream, 0.25% lysostaphin cream, and 0.125% lysostaphin cream. On days 3, 4, and 5 post-instillation, animals were treated with these cream formulations. Two to four hours after the final cream dosing, the animals were sacrificed and S. aureus colonization was measured.

As shown in Table 3a, a final concentration of 0.5% lysostaphin was most effective in eradicating nasal colonization. In this group, all five animals were negative for S. aureus colonization. As the concentration of lysostaphin in the cream was decreased, the average number of colonies in each of the treatment groups, 0.25% and 0.125%, increased. While 0.125% lysostaphin showed no discernable decrease in the average number of colonies compared to the negative control group, the 0.25% lysostaphin cream did alleviate colonization. None of the colonies isolated from lysostaphin treated animals were lysostaphin-resistant.

TABLE 3a
Number of CFUs recovered in 100 μl of
PBS + Tween-20 - 3 doses of cream

| | Negative | 0.5% | 0.25% | 0.125% |
|---|---|---|---|---|
| | Control | Lysostaphin | Lysostaphin | Lysostaphin |
| Animal | Cream | Cream | Cream | Cream |
| 1 | 129 | 0 | 25 | 10 |
| 2 | 906 | 0 | 7 | 680 |
| 3 | 4 | 0 | 532^{a} | 103 |
| 4 | 74 | 0 | 179^{a} | 0 |
| 5 | 34 | 0 | 11 | 556 |
| Average number | 229 | 0 | 150 | 337 |
| of CFU^{b} | | | | |

^{a}These two animals had wounds from fighting around their noses, which may have contributed the high number of colonies recovered.
^{b}CFUs recovered in one fifth of total; volume.

Additional experiments were performed to address the combination of the number of doses of lysostaphin cream and the percent composition of lysostaphin in the lysostaphin cream. The animals were split into seven treatment groups: no treatment, 3 doses of negative control cream, 1 dose of negative control cream, 3 doses of 0.5% lysostaphin cream (˜150 μg lysostaphin per dose), 1 dose of 0.5% lysostaphin cream (˜150 μg lysostaphin), 3 doses of 0.125% lysostaphin cream (˜37.5 μg lysostaphin per dose), and 1 dose of 0.125% lysostaphin cream (˜37.5 μg lysostaphin). Animals were treated with these cream formulations between days 4 and 7 post-instillation. Four to 24 hours after the final cream dosing, the animals were sacrificed and S. aureus colonization was measured. For animals who received no treatment, animals were sacrificed between 4 and 8 days post-instillation.

As shown in Table 3b, a final concentration of 0.5% lysostaphin was again most effective in eradicating nasal colonization. Confirming the results of Table 3a, 3 doses of 0.5% lysostaphin cream completely eradicated S. aureus colonization. A single dose of 0.5% lysostaphin cream significantly reduced colonization. In contrast, the animals that received 0.125% lysostaphin still exhibited nasal S. aureus colonization, with 3 doses being more effective than 1 dose.

TABLE 3b

| | | | | 0.5% | 0.5% | 0.125% | 0.125% |
|---|---|---|---|---|---|---|---|
| | | Placebo | Placebo | Lysostaphin | Lysostaphin | Lysostaphin | Lysostaphin |
| | No | Cream | Cream | Cream | Cream | Cream | Cream |
| — | Treatment | 3 doses | 1 dose | 3 doses | 1 dose | 3 doses | 1 dose |
| Animals | 41/41 | 23/23 | 23/23 | 0/14 | 5/71 | 9/20 | 8/10 |
| Colonized/ | | | | | | | |
| Animals | | | | | | | |
| Tested | | | | | | | |
| Percent | 0 | 0 | 0 | 100 | 93 | 55 | 20 |
| Eradicated | | | | | | | |
| Number of | 10 | 5 | 5 | 3 | 11 | 3 | 2 |
| Experiments | | | | | | | |
| Mean CFU/ | 5262 | 3372 | 6354 | 0 | 8 | 672 | 1006 |
| colonized | | | | | | | |
| nare | | | | | | | |
| Median | 4715 | 2010 | 6790 | 0 | 5 | 165 | 1071 |
| CFU/ | | | | | | | |
| colonized | | | | | | | |
| nare | | | | | | | |

### Example 4

### Comparison of Lysostaphin Cream with Intranasal Mupirocin

Lysostaphin cream was compared to a 2% topical formulation of mupirocin cream (Bactroban) for its ability to eradicate nasal colonization by S. aureus. Twenty cotton rats were instilled with MBT 5040 S. aureus and divided into four treatment groups: untreated negative controls, negative control cream, 0.5% lysostaphin cream, and Bactroban topical. On days 3, 4, and 5 post-instillation, animals were treated with the appropriate cream formulation (or no treatment for group 1). Two to four hours after the last treatment, the animals were sacrificed and nasal colonization measured.

As shown in Table 4, lysostaphin cream eradicated colonization in 4 of 5 animals while Bactroban topical eradicated colonization in all four animals. However, in the Bactroban group, 4 animals had severe scabbing around the nose, perhaps due to irritation caused by the alcohol content of Bactroban topical. Further, because alcohol alone has antibacterial activity, it is difficult to distinguish whether eradication in the Bactroban group was due to the presence of mupirocin or alcohol.

TABLE 4
Number of CFUs recovered in 100 μl of PBS + Tween-20

| | | Negative | 0.5% | Intranasal |
|---|---|---|---|---|
| | | Control | Lysostaphin | Bactroban |
| Animal | Untreated | Cream | Cream | Cream^{b} |
| 1 | 1200 | 804 | 127^{a} | 0 |
| 2 | 95 | 640 | 0 | 0 |
| 3 | 857 | 397 | 0 | 0 |
| 4 | 91 | 170 | 0 | 0 |
| 5 | — | 354 | 0 | — |
| Average number | 560 | 473 | n/a | 0 |
| of CFU | | | | |

^{a}This animal was very sick at the time of sacrifice and may have had an active, systemic infection.
^{b}All four animals had scabbing around the nose.

### Example 5

### Comparison of Lysostaphin Cream with Mupirocin Nasal Ointment

Lysostaphin cream was compared to a 2% mupirocin nasal ointment (Bactroban Nasal) for its ability to eradicate nasal colonization by S. aureus. Twenty cotton rats were instilled with MBT 5040 S. aureus and divided into four treatment groups: negative control cream, 0.5% lysostaphin cream, 0.125% lysostaphin cream, and nasal Bactroban. On days 3, 4, and 5 post-instillation, animals were treated with the appropriate cream formulation. Two to four hours after the last treatment, the animals were sacrificed and nasal colonization measured.

As shown in Table 5a, when given in three doses, both 0.5% lysostaphin cream and Bactroban nasal ointment eradicated nasal colonization in all treated animals. In contrast, 0.125% lysostaphin cream eradicated colonization in 2 out 5 animals. In the remaining three animals, the average number of colonies in those animals was much less than that in the negative control cream group, thus indicating that 0.125% lysostaphin cream can alleviate colonization and in some instances, eradicate it.

TABLE 5a
Number of CFUs recovered in 100 μl of PBS + Tween-20

| | Negative | 0.5% | 0.125% | Nasal |
|---|---|---|---|---|
| | Control | Lysostaphin | Lysostaphin | Bactroban |
| Animal | Cream | Cream | Cream | Ointment |
| 1 | TNTC^{a} | 0^{b} | 47 | 0 |
| 2 | 243 | 0 | 19 | 0 |
| 3 | 84 | 0 | 0 | 0 |
| 4 | TNTC | 0 | 33 | 0 |
| 5 | — | 0 | 0 | 0 |
| Average number | 560 | 0 | 33 | 0 |
| of CFU | | | | |

^{a}For colony counts “too numerous to count,” the CFUs were set at 2000 for the sake of calculating an average.
^{b}This animal died from complications with the anesthesia after only two days of treatments. Its nose was still clear on day 4 (the animal died overnight between days 4 and 5 and only received 2 treatments).

In Example 3 above, 0.125% lysostaphin cream did not affect nasal colonization. The inventors attribute this difference to the increased proficiency of delivering creams by the methods described above. As shown in Table 5b below, a 0.125% lysostaphin cream can be effective in eradicating nasal staphylococcal colonization in as little as two doses.

TABLE 5b
Number of animals colonized with MBT 5040^{a}
following various treatments with 0.125% lysostaphin cream

| Number of | Number of |
|---|---|
| Treatments (Once | Colonized |
| per Day) | Animals |
| No Treatment | 5/5 (2549) |
| 1 | 5/5 (1596) |
| 2 | 0/5 (0) |
| 3 | 0/5 (0) |

^{a}The number in parenthesis is the average number of CFUs per animal.

### Example 6

### A Single Dose of Lysostaphin Cream Eradicates Nasal Colonization by Several Strains of S. aureus

To determine the efficacy of a single dose of 0.5% lysostaphin cream in eradicating S. aureus nasal colonization, cotton rats were instilled with either MBT 5040 (MRSA), Type 5 S. aureus (sensitive to methicillin; MSSA), or Type 8 S. aureus (MSSA). Half of the animals in each group were treated once on day 5 post-instillation with negative control cream and other half of the animals were treated on the same day with 0.5% lysostaphin cream.

As shown in Tables 6a, 6b, 6c, and 6d, 0.5% lysostaphin cream, in a single dose, was effective in eradicating S. aureus nasal colonization.

TABLE 6a
Average number of CFUs per animal

| | Negative | 0.5% Lysostaphin |
|---|---|---|
| S. aureus strain | Control Cream | Cream^{a} |
| MBT 5040^{b} | 1000 | 0 |
| Type 5 MSSA | >2000 | 0 |
| Type 8 MSSA | 1400 | 0 |

^{a}Bacteria were eliminated by four hours post-treatment.
^{b}Data represents the average of ten experiments.

TABLE 6b
Number of animals colonized^{a}

| | Negative | 0.5% Lysostaphin |
|---|---|---|
| S. aureus strain | Control Cream | Cream |
| MBT 5040^{b} | 45/45 (≈1000) | 0/45 (0) |
| MSSA^{c} | 10/10 (≈2000) | 0/10 (0) |

^{a}The number in parenthesis is the average number of CFUs per animal.
^{b}Represents the results of 7 experiments.
^{c}Represents the results of 2 experiments. S. aureus Type 5 (ATCC No. 49521) was used in 5 rats. S. aureus Type 8 (ATCC No. 12605) was used in the other 5 rats.

TABLE 6c
Number of animals colonized^{a}

| | Negative | 0.5% Lysostaphin |
|---|---|---|
| S. aureus strain | Control Cream | Cream |
| MBT 5040 | 5/5 (627) | 0/5 (0) |
| Mupirocin Resistant^{b} | 5/5 (254) | 0/5 (0) |

^{a}The number in parenthesis is the average number of CFUs per animal.
^{b}S. aureus strain SA 3865, containing a mupirocin resistance plasmid, was used for this experiment.

TABLE 6d

| | S. aureus strains | | | |
|---|---|---|---|---|
| | S. aureus | S. aureus | | SA 3865 |
| | Type 5 | Type 8 | MRSA 12/12¹ | Mup^{R2} |
| Animals Colonized/ | 5/5 | 5/5 | 5/5 | 5/5 |
| Animals Tested | | | | |
| CONTROL GROUP | | | | |
| Mean Colonization | >10,000 | 5418 | 567 | 268 |
| CONTROL GROUP | | | | |
| Animals Colonized/ | 0/5 | 0/5 | 0/5 | 0/5 |
| Animals Tested | | | | |
| LYSOSTAPHIN | | | | |
| TREATED GROUP | | | | |
| Mean Colonization | 0 | 0 | 0 | 0 |
| LYSOSTAPHIN | | | | |
| TREATED GROUP | | | | |

¹Fresh clinical isolate from WRAMC.
²See Morton et al., Antimicrob. Agents Chemother. 39:1272-80 (1995).

As demonstrated in the above examples, the inventors have greatly improved over the previous studies

### Example 7

### A Single Dose of Lysostaphin Cream is More Effective than Mupirocin Nasal or Nisin In Vivo

A single dose of 0.5% lysostaphin cream was tested against a single dose of 2% mupirocin ointment and a single dose of two concentrations of nisin cream. Nisin is a lantibiotic with good in vitro anti-staphylococcal activity even when formulated in cream. Cotton rats were instilled with MBT 5040 S. aureus and, on day 5 post-instillation, were treated with one of the following: 0.5% lysostaphin cream, 2% mupirocin ointment (Bactroban), 5% nisin cream, or 0.5% nisin cream. Twenty four hours after treatment, the animals were sacrificed and nasal colonization was measured. As shown in Table 7, only the single dose of 0.5% lysostaphin was able to eradicate staphylococcal colonization. Each of the other treatment groups contained colonized animals. Thus, when given in a single dose, lysostaphin cream is more effective at eradicating nasal colonization than mupirocin or nisin.

TABLE 7

| | Number of Animals Colonized 24 | Average number |
|---|---|---|
| Nasal Cream | hours Post-Treatment | of CFU per nose |
| Negative Control | 5/5 | 4140 |
| 0.5% | 0/5 | 0 |
| Lysostaphin | | |
| 2% Mupirocin | 3/5 | 70 |
| 5% Nisin | 4/4 | 4311 |
| 0.5% Nisin | 5/5 | 7320 |

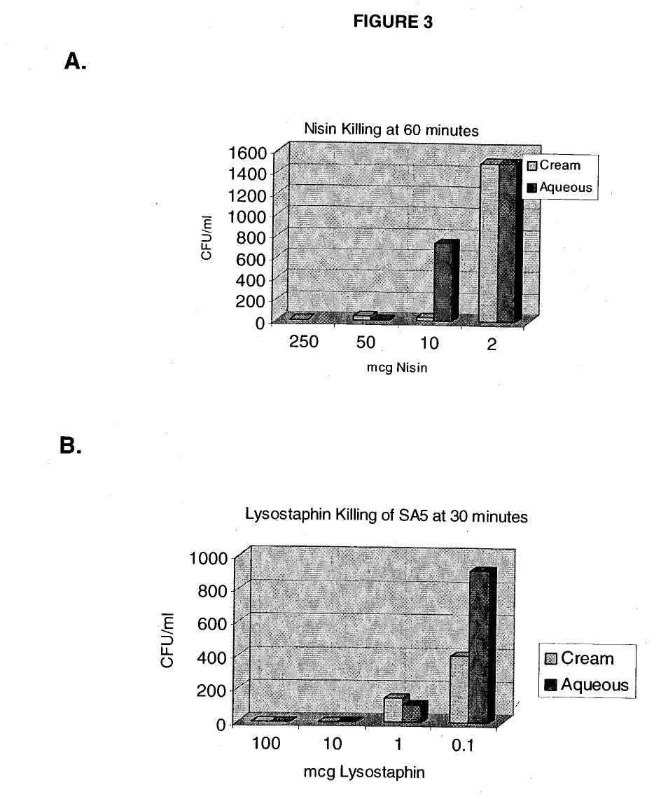

We note that both nisin cream formulations have good anti-staphylococcal activity in vitro (FIGS. 3A and 3B) and yet did not alleviate nasal colonization in vivo. When further tested, the nisin creams lost anti-staphylococcal activity within two hours of instillation in the nose, either due to inactivation of nisin activity or sequestering of the nisin molecules. Why the nisin cream was effective in vitro and not in vivo remains to be determined, but these data exemplify how in vitro studies do not always reflect the interactions that occur in vivo.

### Example 8

### Animals Receiving Two Doses of Lysostaphin Cream Remain Colonization-Free for at Least One Week Post-Administration

As discussed above, 0.5% lysostaphin cream can effectively eradicate nasal staphylococcal colonization. To determine how long this eradication may last, three cotton rats were instilled with MBT 5040 S. aureus. On days 5 and 6 post-instillation, the animals were given one dose of 0.5% lysostaphin cream. In parallel, five cotton rats were also instilled and not treated. At one week following the instillation of lysostaphin, the animals treated with lysostaphin cream had no colonies present in the nares. In contrast, the five untreated animals had an average of 2200 CFU per nose at the time of treatment while five other untreated animals had an average of 1755 CFU per nose at the time of sacrifice. Thus, animals treated with two doses of 0.5% lysostaphin cream remained free from S. aureus nasal colonization for at least one week post-administration.

### Example 9

### A Single Dose of Lysostaphin Cream Eradicates Staphylococcal Colonization 4 hours Post-Administration and Remains Active in the Nares for at Least Forty Eight Hours

To determine how quickly lysostaphin eradicates nasal colonization, ten cotton rats were instilled with MBT 5040 S. aureus. Six days post-instillation, five animals were treated with a single dose of 0.5% lysostaphin cream and the other five treated with control cream. All animals were sacrificed 4 hours after administration of the cream and their noses analyzed for colonization. As shown in Table 8a, lysostaphin cream eradicated staphylococcal colonization in as little as four hours post-administration.

TABLE 8a
Number of animals colonized with MBT 5040 S. aureus

| | Number of Animals Colonized 4 |
|---|---|
| Nasal Cream | hours Post-Treatment¹ |
| Negative Control | 5/5 (5446) |
| 0.5% Lysostaphin | 0/5 (0) |

¹The number in parenthesis is the average number of CFUs per animal.

To determine how long lysostaphin, when administered in a cream formulation, remains active in the nares, a single dose of 2% lysostaphin cream was instilled in cotton rat noses 48 hours, 24 hours, and 8 hours prior to instillation of 10⁹ CFU of S. aureus MBT 5040. Control animals received control cream, without lysostaphin, 8 hours prior to receiving bacteria. Six days after introduction of the bacteria, each of the 5 animals per experimental group were sacrificed and the noses checked for S. aureus colonization. The results of this experiment are shown in Table 8b.

TABLE 8b
Number of animals colonized with MBT 5040 S. aureus

| | Control | 2% | 2% | 2% |
|---|---|---|---|---|
| | Cream | Lysostaphin | Lysostaphin | Lysostaphin |
| Animal | 8 hours prior | 8 hours prior | 24 hours prior | 48 hours prior |
| 1 | >1200 | 422 | >1700 | >1600 |
| 2 | 1033 | 0 | 0 | 4 |
| 3 | 804 | 0 | 1 | 72 |
| 4 | >2000 | 0 | 1 | 22 |
| 5 | 409 | 0 | 0 | 7 |

In 4 out of 5 animals in each group, lysostaphin pre-instillation dramatically blocked or alleviated the colonization. Though this effect was greatest in rats treated 8 hours prior to receiving bacteria, the 2% lysostaphin cream still alleviated colonization when delivered 48 hours before instillation of bacteria.

Thus, not only does lysostaphin, when administered in a viscous formulation such as a cream, quickly eradicate staphylococcal colonization of the nares, it remains active in the nares for at least 48 hours after administration.

### Example 10

### USP Grade Lysostaphin Cream

A lysostaphin cream formulation was prepared as described in Example 2 (“original cream”) above, using the same components in the same percentage amounts. In the USP cream, however, each ingredient used was U.S. Pat. No. grade (or EP European Pharmacopeia or DMF, Drug Master File) meeting particular certification standards for clinical use. In addition, aluminum stearate was substituted with zinc stearate in the formulation. Using this USP cream formulation, a 0.5% and a 2% lysostaphin cream were produced and these USP cream formulations were compared to the cream formulation of Example 2 for effectiveness.

Five cotton rats per experimental group were instilled with MBT 5040 S. aureus. Five days after introduction of bacteria into the anterior nares, rats were given control cream, 0.5% lysostaphin in original cream, 0.5% lysostaphin in USP cream, 2% lysostaphin in original cream, or 2% lysostaphin in USP cream according to each experimental group. All animals were sacrificed 24 hours later and analyzed for S. aureus nasal colonization. As shown in Table 9, the USP grade lysostaphin cream was just as effective in eradicating nasal colonization as the original lysostaphin cream.

TABLE 9
Number of animals colonized with MBT 5040 S. aureus

| | Number of Animals Colonized 24 |
|---|---|
| Nasal Cream | hours Post-Treatment¹ |
| Negative Control | 5/5 (>2000) |
| 0.5% Lysostaphin Original | 0/5 (0) |
| 0.5% Lysostaphin USP | 0/5 (0) |
| 2% Lysostaphin Original | 0/5 (0) |
| 2% Lysostaphin USP | 0/5 (0) |

¹The number in parenthesis is the average number of CFUs per animal.

### Example 11

### Homogenous Lysostaphin Cream Also Eradicates Nasal Colonization

As discussed above, the lysostaphin used in Examples 1-10 was Ambicin L (Ambi, Inc.). Ambicin L lysostaphin is a preparation of heterogenous forms of lysostaphin. Specifically, the enzyme molecules in Ambicin L start at different amino acids in the lysostaphin sequence due to proteolytic processing of the recombinant pro-enzyme. Thus, Ambicin L represents a mixture of different species of lysostaphin molecules. To determine whether a homologous preparation of lysostaphin would also eradicate or alleviate nasal S. aureus colonization, recombinant lysostaphin was prepared such that every lysostaphin molecule in the preparation began with the first threonine in the lysostaphin sequence. Provisional patent application Serial No. 60/341,804, and related non-provisional application, Truncated Lysostaphin Molecule With Enhanced Staphylolyticd Activity, submitted herewith, both of which are specifically incorporated by reference, contain further details as to the cloning and preparation of this homogenous lysostaphin. This recombinant homogenous lysostaphin was used to prepare a 0.5% lysostaphin cream, as described above.

Cotton rats were instilled with MBT 5040 S. aureus and divided into three experimental groups: negative control cream, 0.5% Ambicin L lysostaphin cream, and 0.5% homogenous lysostaphin cream. Each animal was then treated with a single dose of cream preparation on day 6 post-instillation, according to these groups. As shown in Table 11 below, homogenous lysostaphin also eradicated nasal colonization.

TABLE 11
Number of animals colonized with MBT 5040 S. aureus

| | Number of Animals |
|---|---|
| Treatment | Colonzied¹ |
| Negative Control Cream | 4/4 (8875) |
| 0.5% Ambicin Lysostaphin Cream | 0/5 (0) |
| 0.5% Homogenous Lysostaphin | 0/5 (0) |
| Cream | |

¹The number in parenthesis is the average number of CFUs per animal.

Surprisingly, lysostaphin resistant S. aureus has never been recovered from the nose of a lysostaphin treated animal in over sixty experiments conducted with various doses and formulations of lysostaphin (data not shown). In contrast, lysostaphin resistance has been documented in instances where lysostaphin is given systemically to treat a systemic infection (17). An explanation for the lack of lysostaphin-resistant S. aureus being isolated from lysostaphin-treated nares maybe found in the discovery that when a lysostaphin-resistant strain of S. aureus isolated in vitro from MBT 5040 by treatment of the S. aureus with sub-MIC doses of lysostaphin was instilled into the nares of five cotton rats, only one animal became nasally colonized and in this animal, the bacteria remained lysostaphin resistant. This suggests that mutations that confer lysostaphin resistance negatively affect the bacterium's ability to colonize the nares. To test this hypothesis, a lysostaphin-resistant variant of MBT 5040 (MBT 5040 LysoR, MIC >32 μg/ml) that was isolated in vitro was instilled in cotton rat nares at 10⁹. Only one of five cotton rats instilled with MBT 5040 LysoR became colonized with this lysostaphin-resistant variant and this animal only had 200 CFUs recovered from its nose on day 7 as compared to an average of ˜5000 CFUs recovered from the noses of animals instilled with wild type MBT 5040 in the same experiment.

To further explore the relationship between lysostaphin-resistance and nasal colonization, treatment of S. aureus strain MRSA 12/12 nasal colonization with a single dose of 0.5% lysostaphin cream was also examined. MRSA 12/12 colonized the cotton rat nares (Table 6d), and a single treatment of 0.5% lysostaphin cream also eradicated nasal colonization by this strain. Nasal supernatant from treated animals was plated on both TSA/NaCl+nafcillin and TSA/NaCl alone since when MRSA become lysostaphin-resistant they revert to MSSA (17). Forty three colonies that grew on TSA without antibiotic were examined for identity and none were found to be S. aureus by the Staphyloslide™ latex test, i.e., no lysostaphin-resistant S. aureus MRSA 12/12 was recovered from the noses of S. aureus instilled animals treated with a single dose of 0.5% lysostaphin cream or from the nose of any animal treated with lysostaphin cream regardless of what strain of S. aureus was instilled (Table 6d).

As discussed above, there is only one commercially available product for eradication of S. aureus nasal colonization, Bactroban Nasal. Given the growing resistance to mupirocin, a method for treating nasal colonization, a major site providing a reservoir for potential infections in the host, that does not generate resistance to the active agent, provides an important added advantage over the current treatment.

The toxicity of lysostaphin cream in vivo was also addressed. Cotton rats were given one dose per day of 0.5% lysostaphin cream intranasally for three consecutive days. Two weeks later, the rats were treated again with one dose of 0.5% lysostaphin intranasally per day for three days. Two weeks later the rats were given a final, single dose of 0.5% lysostaphin intranasally and sacrificed 48 hours after that final dose. This lysostaphin dosing schedule induced a strong antibody response in all animals tested. Nasal tissues were then harvested and analyzed for histological abnormalities. All tissues harvested from animals treated with 7 doses of 0.5% lysostaphin cream were within normal limits. Thus, the 0.5% concentration used in the above examples is non-toxic in vivo, even in the presence of anti-lysostaphin antibodies.

In a second set of toxicity studies, 0.5% lysostaphin cream was applied to the noses of rabbits once a day for fourteen days. At the end of this time, the histology of the noses were examined and found to be normal.

### Example 12

### Lysostaphin Cream Produced Under GMP Conditions Eradicates S. aureus and Demonstrates Excellent Stability

Processes have been developed to produce USP-grade lysostaphin cream, as described in Example 10, under good manufacturing procedures (GMP) in commercially viable quantities. This lysostaphin cream was made with a homogenous form of recombinant lysostaphin produced in Lactococcus lactis, which is discussed above in Example 11. The GMP lysostaphin cream was further subjected to accelerated stability testing by storing the lysostaphin cream at room temperature with monitoring to simulate one year storage at room temperature. The lysostaphin cream was examined periodically for potency, purity, pH, appearance, microbial growth, and rehology.

Cotton rats were instilled with MBT 5040 S. aureus and divided into three experimental groups: negative control cream, 0.5% GMP lysostaphin cream, and 0.5% GMP lysostaphin cream subjected to accelerated stability testing. Each animal was then treated with a single dose of each cream preparation on day 6 post-instillation, according to these groups. As shown in Table 10a below, GMP lysostaphin cream eradicated nasal colonization and little if any anti-staphylococcal activity was lost from the sample subjected to accelerated stability testing. Table 10b demonstrates that both 0.5% and 1% lysostaphin GMP cream can dramatically reduce staphylococcal colonization even 7 days post-instillation.

TABLE 10a
Number of animals colonized with MBT 5040 S. aureus

| | Number of Animals |
|---|---|
| Treatment | Colonized¹ |
| Negative Control Cream | 5/5 (2074) |
| 0.5% GMP Lysostaphin Cream | 0/5 (0) |
| 0.5% Stability-tested Lysostaphin | 1/5 (5)² |
| Cream | |

¹The number in parenthesis is the average number of CFUs per animal colonized.
²One S. aureus colony was recovered from one animal treated with stability tested GMP lysostaphin cream.

TABLE 10b
A direct comparison of GMP 0.5% and 1% lysostaphin creams.
Number of animals colonized with MBT 5040 S. aureus

| | | Average CFUs |
|---|---|---|
| | Number of Animals | Recovered per |
| Treatment | Colonized⁽¹⁾ | Nose |
| Placebo Cream | 10/10⁽¹⁾ | 766 |
| 0.5% GMP Lysostaphin Cream | 5/10 | 8⁽²⁾ |
| 1% GMP Lysostaphin Cream | 1/10 | 5⁽²⁾ |

⁽¹⁾Animals nasally colonized over animals tested.
⁽²⁾Actual CFUs recovered range from 1-3. The dilution factor was 5.

In this particular experiment, 1% GMP lysostaphin cream was more effective than 0.5% GMP lysostaphin cream.

### Example 13

### The Phage Enzyme Phi 11 Hydrolase Synergizes with Lysostaphin

The lytic S. aureus phage phi 11 produces an enzyme that has some anti-staphylococcal properties on its own. As shown below, this enzyme, phi 11 hydrolase, demonstrated synergy with lysostaphin. Thus, it may be advantageous to add purified phi 11 hydrolase to a lysostaphin cream to increase its over all effectiveness and perhaps decrease the amount of lysostaphin needed for an effective product.

A checker board synergy assay was used to observe the effect of lysostaphin and phi 11 hydrolase in combination on staphylococci. In a 96-well assay plate, two-fold dilutions of lysostaphin ranging from 250 ng/ml to 0.25 ng/ml were prepared as follows. Fifty microliters of Cation-adjusted Mueller-Hinton Broth+2%NaCl+0.1% BSA (CAMHB++ media) was added to columns 1-11, panning rows A-H. A stock solution of 1 μg/ml lysostaphin in CAMHB++ media was prepared. One hundred microliters of this lysostaphin stock was added to column 12, spanning rows A-H. Fifty microliters of lystaphin stock was transferred from column 12 to column 11 and mixed by pipetting. Fifty microliters of diluent from column 11 was transferred to column 10 and mixed and so on, stopping at column 2. Fifty microliters of lysostaphin diluent was removed from column 2 and discarded.

In a separate plate, 2-fold dilutions of phi 11 hydrolase in CAMHB++media were prepared. First, a stock solution of 10 μg/ml phi 11 hydrolase in CAMHB++ media was prepared. Seventy five microliters of CAMHB++ media was added to rows A-G, spanning columns 1-12. One hundred and fifty microliters of the stock solution was added to row H, spanning columns 1-12. Seventy five microliters of hydrolase stock was transferred from row H to row G and mixed by pipetting. Seventy five microliters of diluent from row G was transferred to row F and mixed and so on, stopping at row B. Seventy five microliters of hydrolase diluent was removed from row B and discarded. Fifty microliters of hydrolase diluent was transferred from the second plate to the assay plate, starting with row A of the second plate and proceeding to row H. Approximately 10⁵ S. aureus bacteria in a 100 μl volume of CAMHB++ media were added to each well of the assay plate, spanning columns 1-12 and rows A-H. Assay plates were incubated overnight with shaking at 37° C. The O.D. at 650 nm was then measured and 2 μl of a 1.6 μg/pl stock of lysostaphin was added to suspected lysostaphin resistance outgrowths. The assay plate was again incubated overnight under the same conditions and the O.D. checked the following day.

Table 12 depicts the results of the assay for the combination of lysostaphin and phi 11 hydrolase. Specifically, as the concentration of hydrolase increased, the concentration of lysostaphin needed to inhibit growth decreased.

TABLE 12
Synergy between lysostaphin and phi 11 hydrolase.

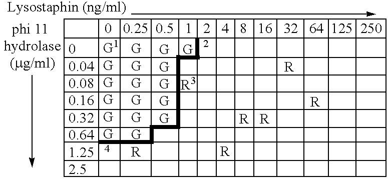

¹G = Normal growth
²Blank = no growth
^{R}= resistance outgrowth as determined by addition of 20μ/ml lysostaphin for 4 hrs following initial overnight incubation. Wells in which the optical density stays the same or increases are lysostaphin-resistant outgrowths.
⁴Bold line indicates area where synergy between lysostaphin and phi 11 hydrolase is evident.

### Example 14

### Lysostaphin Formulations With An Additional Antibacterial Agent Inhibit Lysostaphin-Resistant Outgrowth

When lysostaphin is tested in a standard “Minimum Inhibitory Concentration” (MIC) assay, outgrowth of S. aureus is sometime observed above the MIC for almost all strains of S. aureus examined. On further testing, these outgrowth wells are found to contain lysostaphin-resistant S. aureus with MICs many dilutions above the MIC of the parental strain. While lysostaphin-resistant S. aureus has never been recovered from the nose of a cotton rat treated with lysostaphin cream, additional agents may ensure that lysostaphin-resistant strains do not arise in larger animals, or in a larger pool of patients treated with a composition of the invention. This Example illustrates that the addition of the antibacterial agent bacitracin, eliminates lysostaphin-resistant outgrowths in vitro, and may likewise reduce or eliminate resistant outgrowths in vivo. In particular, this Example illustrates that the addition of bacitracin to nasal lysostaphin formulations at concentrations of bacitracin below the MIC of bacitracin for a particular strain of S. aureus inhibits outgrowth of lysostaphin-resistant S. aureus above the lysostaphin MIC for that strain. Of course, nasal formulations comprising antibacterial agents above the MIC for that particular agent are also within the scope of this invention.

Table 13 depicts one such experiment for a strain of S. aureus (ATCC 49521). In this experiment a NCCLS standard MIC is conducted with the modification of adding 0.1% BSA to the assay. Rows 1-4 are lysostaphin MICs conducted in the absence of bacitracin while rows 5-8 are lysostaphin MICs conducted in the presence of bacitracin (5 μg/ml).

TABLE 13
Addition of sub-MIC bacitracin inhibits outgrowth
of lysostaphin resistance

| | Lysostaphin | | | | | | | | | | | |
|---|---|---|---|---|---|---|---|---|---|---|---|---|
| | ng/ml | | | | | | | | | | | |
| Baci- | | | | | | | | | | | | |
| tracin (5 | | | | | | | | | | | | |
| μg/ml) | 0 | 0.25 | 0.5 | 1 | 2 | 4 | 8 | 16 | 32 | 64 | 125 | 250 |
| − | G¹ | G | G | G | ² | R³ | R | R | R | | | R |
| − | G | G | G | G | R | | | R | R | | R | |
| − | G | G | G | G | R | | R | | R | | R | |
| − | G | G | G | G | | R | R | R | R | | | |
| + | G | G | G | G | | | | | | | | |
| + | G | G | G | | | | | | | | | |
| + | G | G | G | | | | | | | | | |
| + | G | G | G | G | | | | | | | | |

¹G = Normal growth
² Blank = no growth
³R = resistance outgrowth as determined by addition of 20 μg/ml lysostaphin for 4 hrs following initial overnight incubation. Wells in which the optical density stays the same or increases are lysostaphin-resistant outgrowths.

Addition of sub-MIC bacitracin to lysostaphin MIC assays inhibited outgrowth of lysostaphin-resistant S. aureus in most S. aureus strains tested. As mentioned above, lysostaphin-resistant S. aureus have not been isolated from the noses of cotton rats, so this model is not helpful in demonstrating the same effect in vivo. Nevertheless, this finding suggests that it may be advantageous to add bacitracin or other antibacterial agent to a lysostaphin cream to help prevent outgrowth of lysostaphin-resistant strains.

### Example 15

### Lysostaphin Formulations With Lysostaphin or Nisin

Lysostaphin MIC assays were conducted in the presence or absence of subinhibitory concentrations (four-fold dilution below MIC) of either bacitracin or nisin to determine if either substance prevented the outgrowth of lysostaphin resistance in these assays. This is as previously described in Example 14, above.

TABLE 14
Addition of sub-MIC bacitracin or nisin

| | | Lysostaphin + | Lysostaphin + |
|---|---|---|---|
| Treatment | Lysostaphin alone | nisin | bacitracin |
| ATCC 49521 | 31^{a} | 19 | 0 |
| SA5 USU | 14 | 18 | 0 |
| SA5 Sam | 18 | 28 | 3 |
| SA8 Sam | 58 | 29 | 0 |

^{a}Percentage of wells above lysostaphin MIC that have outgrowth of lysostaphin-resistance.

Thus, the presence of subinhibitory concentrations of bacitracin strongly inhibit the outgrowth of lysostaphin resistance. Note, the MIC of lysostaphin-resistant S. aureus for bacitracin is the same as it is for the lysostaphin sensitive parental strain (data not shown), so it is not merely the lysostaphin-resistant S. aureus becoming more bacitracin sensitive that leads to this phenomenon.

### CONCLUSION

Thus, Examples 1 and 2 show that lysostaphin in a cream formulation is more effective at eradicating and alleviating nasal staphylococcal colonization than lysostaphin in PBS. Example 2 also demonstrates that lysostaphin activity can remain in the nares for an extended period of time and that proteinase K can inactivate lysostaphin. Further, Example 2 demonstrates that lysostaphin eliminates S. aureus in the nose rather than ex vivio during sampling. Example 3 demonstrates that, when compared to 0.25% and 0.125% lysostaphin creams, 0.5% lysostaphin cream worked better to eradicate and alleviate staphylococcal colonization in the nares. Examples 4 and 5 show that, when given in three doses, both 0.5% lysostaphin cream and 2% mupirocin cream or ointment can eradicate nasal colonization. Example 6 shows that lysostaphin cream eradicates S. aureus nasal colonization with a single dose every time attempted and against several strains of S. aureus. Example 7 demonstrates that lysostaphin cream is more effective in eradicating nasal colonization in a single dose as compared to single doses of mupirocin or nisin. Example 8 shows that lysostaphin-treated noses can remain free of S. aureus recolonization for at least a week after administration of lysostaphin cream. Example 9 demonstrates that lysostaphin cream can block and alleviate S. aureus colonization for up to 24 hours prior to instillation of bacteria. At 48 hours pre-instillation, lysostaphin continues to decrease colonization in the nose. Examples 10 and 11 demonstrate that USP-grade lysostaphin cream and stability tested USP-grade lystostaphin cream made under GMP conditions are effective at eradicating or alleviating S. aureus colonization in the nose. Example 12 demonstrates that a homogenous preparation of lysostaphin in a cream formulation works just as well to eradicate nasal colonization as a lysostaphin cream containing heterologous forms of lysostaphin. Finally, Example 13 demonstrates a synergy between lysostaphin and phi 11 hydrolase, suggesting that it may be advantageous to add phi11 hydrolase to lysostaphin cream to enhance its effectiveness.

In sum, a viscous lysostaphin intranasal, such as a lysostaphin cream, is more effective in eradicating or alleviating nasal staphylococcal colonization than a single dose of alternate treatments currently available such as Bactroban. Lysostaphin cream eradicates and alleviates nasal colonization very quickly after the first administration, remains active for at least 48 hours after administration, and is effective in as little as one dose. Lysostaphin-resistant S. aureus was not detected in any of the above Examples, indicating that the instant invention offers an added benefit of eradicating nasal colonization without producing resistant strains that may be spread into the community. In contrast, mupirocin resistance among S. aureus strains has become increasingly problematic and is found intranasally (26). Lastly, the 0.5% concentration used in the majority of the examples is not toxic in vivo.

One of skill in the art would realize that the lysostaphin intranasals that eradicate, alleviate, or block staphylococcal nasal colonization are not limited only to recombinant lysostaphin. Other forms of lysostaphin, as discussed above, may also be used in lysostaphin creams. Further, lysostaphin creams can not only eradicate, but also alleviate colonization of the nares by S. aureus. The usefulness of such other lysostaphin creams will be determined by comparison to control groups of cotton rats treated with a negative control cream to ensure that lysostaphin causes the measured effect.

The following publications are hereby specifically incorporated by reference:

1. Aly, R. and S. Levit. 1987. Adherence of Staphylococcus aureus to squamous epithelium: role of fibronectin and teichoic acid. Rev. Infect. Dis. 9:S341-S350.

2. Aly, R., H. R. Shinefeld, C. Litz and H. I. Maibach. 1980. Role of teichoic acid in the binding of Staphylococcus aureus to nasal epithelial cells. J. Infect. Dis. 141:463-465.

3. Aly, R., H. I. Shinefield, W. G. Strauss and H. I. Maibach. 1977. Bacterial adherence to nasal mucosal cells. Infect. Immun. 17:546-549.

4. Anderson, I. and, D. F. Proctor. 1983. Measurement of nasal mucociliary clearance. Europ. J Respir. Dis. 64:37-40.

5. Baddour, L. M., C. Lowrance, A. Albus, J. H. Lowrance, S. K. Anderson and J. C. Lee. 1992. Staphylococcus aureus microcapsule expression attenuates bacterial virulence in a rat model of experimental endocarditis. J. Infect. Dis. 165:749-753.

6. Beachy, E. H. and H. S. Courtney. 1987. Bacterial adherence: the attachment of group A streptococci to mucosal surfaces. Rev. Infect. Dis. 9:S475-S481.

7. Bibel, D. J., R. Aly, H. R. Shinefield, H. I. Maibach and W. G. Strauss. 1982. Importance of the keratinized epithelial cell in bacterial adherence. J. Invest. Dermatol. 79:250-253.

8. Braman, J., C. Papworth, and A. Greener. 1996. Site-directed mutagenesis using double-stranded plasmid DNA templates. Methods Mol. Biol. 57:31-44.

9. Buckely, R. and R. Schiff. 1991. The use of intravenous immune globulin in immunodeficiency diseases. New Engl. J. Med. 325:110-115.

10. Carruthers, M. M. and W. J. Kabat. 1983. Mediation of staphylococcal adherence to mucosal cells by lipoteichoic acid. Infect. Immun. 40:444-446.

11. Chan, R. C. Y., G. Reid, R. T. Irvin, A. W. Bruce and J. W. Costerton. 1985. Competitive exclusion of uropathogens from human uroepithelial cells by Lactobacillus whole cell and cell wall fragments. Infect. Immun. 47:84-89.

12. Chang, F. Y., N. Singh, T. Gayowski, S. D. Drenning, M. M. Wagener and I. R. Marino. 1998. Staphylococcus aureus nasal colonization and association with infections in liver transplant recipients. Transplantation 65:1169-1172.

13. Chapoutot, C., G.-P. Pageaux, P. F. Perrigault, Z. Joomaye, P. Perney, H. Jean-Pierre, O. Jouquet, P. Blanc and D. Larrey. 1999. Staphylococcus aureus nasal carriage in 104 cirrhotic and control patients A prospective study. J. Hepatol. 30:249-253.

14. Chatfield, C., W. O'Neill, R. Cooke, K. McGhee, M. Issack, M. Rahman and W. Noble. 1994. Mupirocin-resistant Staphylococcus aureus in a specialist school population. J. Hosp. Infect. 26:273-278.

15. Chugh, T. D., G. J. Burns, H. J. Shuhaiber and G. M. Bahr. 1990. Adherence of Staphylococcus epidermidis to fibrin-platelet clots in vitro mediated by lipoteichoic acid. Infect. Immun. 58:315-319.

16. Chugh, T. D., G. J. Burns, H. J. Shuhaiber and E. A. Bishbishi. 1989. Adherence of Staphylococcus epidermis to human epithelial cells: evidence for lipase-sensitive adhesion and glycoprotein receptor. Current Micro. 18:109-112.

17. Climo M W, K. Ehlert, and G. L. Archer. 2001. Mechanism and suppression of lysostaphin resistance in oxacillin-resistant Staphylococcus aureus. Antimicrob Agents Chemother. 45:1431-1437.

18. Cookson, B. 1990. Mupirocin resistance in staphylococci. J. Antimicrob. Chemother. 25:497-503.

19. Corbella, X., M. Dominguez, M. Pujol, J. Aytas, M. Sendra, R. Pallares, J. Ariza and F. Gudiol. 1997. Staphylococcus aureus nasal carriage as a marker for subsequent staphylococcal infections in intensive care units patients. Eur. J. Clin. Microbial. Infect. Dis. 16:351-357.

20. Dawson, S., L. Finn, J. McCulloch, S. Kilvington and b. Lewis. 1994. Mupirocin-resistant MRSA. J. Hosp. Infect. 28:75-78.

21. De Kimpe, S. J., M. Kengatharan, C. Thiemermann and J. R. Vane. 1995. The cell wall components peptidoglycan and lipoteichoic acid from Staphylococcus aureus act in synergy to cause shock and multiple organ failure. PNAS-USA 92:10359-10363.

22. Doebbeling, B. N., D. Breneman, H. Neu, R. Aly, B. Yangco, H. Holley, R. Marsh, M. Pfaller, J. McGowan, B. Scully, D. Reagan and R. Wenzel. 1993. Elimination of Staphylococcus aureus nasal carriage in health care workers: analysis of six clinical trials with calcium mupirocin ointment. Clin. Infect. Dis. 17:466-474.

23. Fierobe, L., D. Decre, C. Muller, J.-C. Lucet, J.-P. Marmuse, J. Mantz and J.-M. Demonts. 1999. Methicillin-resistant Staphylococcus aureus as a causative agent of postoperative intra-abdominal infection: relation to nasal colonization. Clin. Infect. Dis. 29:1231-1238.

24. Foster, T. J. and D. McDevitt. 1994. Surface-associated proteins of Staphylococcus aureus: their possible role in virulence. FEMS Microbiol. Lett. 118:199-205.

25. Frebourg, N., B. Cauliez and J.-F. Lemeland. 1999. Evidence for nasal carriage of methicillin-resistant staphylococci colonizing intravascular devices. J. Clin. Micro. 37:1182-1185.

26. Fung, S., S. O'Grady, C. Kennedy, H. Dedier, I. Campbell, and J. Conly. 2000. The Utility of Polysproin Ointment in Eradication of methicillin-resistant S. aureus colonization: a pilot study. Infect. Control Hosp. Epidemiol. 21:653-655.

27. Granato, D., F. Perotti, I. Masserey, M. Rouvet, M. Golliard, A. Servin and D. Brassart. 1999. Cell surface-associated lipoteichoic acid acts as an adhesion factor for attachment of Lactobacillus johnsonii La1 to human enterocyte-like Caco-2 cells. Appl. Enviro. Micro. 65:1071-1077.

28. Harris, R. L., A. W. Nunnery, and H. D. Riley, Jr. 1967. Effect of Lysostaphin on Staphylococcal Carriage in Infants and Children. Antimicrob. Agents & Chemother. 110-112.

29. Herrmann, M., J. Hartleib, B. Kehrel, R. R. Montgomery, J. J. Sixma and G. Peters. 1997. Interaction of von Willebrand factor with Staphylococcus aureus. J. Infect. Dis. 176:984-991.

30. Herrmann, M., Q. J. Lai, R. M. Albrecht, D. F. Mosher and R. A. Proctor. 1993. Adhesion of Staphylococcus aureus to surface-bound platelets: role of fibrinogen/fibrin and platelet integrins. J. Infect. Dis. 167:312-322.

31. Hoefnagels-Schuermans, A., W. E. Petermans, M. Jorissen, S. Van Lierde, J. van den Oord, R. De Vos and J. Van Eldere. 1999. Staphylococcus aureus adherence to nasal epithelial cells in a physiological in vitro model. In Vitro Cell. Dev. Biol.-Animal. 35:472-480.

32. Johnson S, C. Oliver, G. A. Prince, V. G. Hemming, D. S. Pfarr, S. C. Wang, M. Dormitzer, J. O'Grady, S. Koenig, J. K. Tamura, R. Woods, G. Bansal, D. Couchenour, E. Tsao, W. C. Hall, and J. F. Young. 1997 Development of a humanized monoclonal antibody (Medi 493) with potent in vitro and in vivo activity against respiratory syncytial virus. J. Infect. Dis. 176:1215-1224.

33. Kiser, K. B., J. M. Cantey-Kiser and J. C. Lee. 1999. Development and characterization of a Staphylococcus aureus nasal colonization model in mice. Infect. Immun. 67:5001-5006.

34. Kluytmans, J., A. van Belkum and H. Verbrugh. 1997. Nasal carriage of Staphylococcus aureus: epidemiology, underlying mechanisms, and associated risks. Clin. Micro. Rev. 10:505-520.

35. Kluytmans, J. A., J. W. Mouton, M. VandenBergh, M.-J. Manders, A. Maat, J. Wagenvoort, M. Michel and H. Verbrugh. 1996. Reduction of surgical-site infections in cardiothoracic surgery by elimination of nasal carriage of Staphylococcus aureus. Infect. Control. Hosp. Epidem. 17:780-785.

36. Kublick, H. and M. T. Vidgren. 1998. Nasal delivery systems and their effect on deposition and absorption. Advan. Drug Deliv. Rev. 29:157-177.

37. Lee, Y.-L., T. Cesario, A. Pax, C. Tran, A. Ghouri and L. Thrupp. 1999. Nasal colonization by Staphylococcus aureus in active, independent community seniors. Age Ageing. 28:229-232.

38. Lees A; F. Finkelman; J. K. Inman; K. Witherspoon; P. Johnson; J. Kennedy; and J. J. Mond. 1994. Enhanced immunogenicity of protein-dextran conjugates: I. Rapid stimulation of enhanced antibody responses to poorly immunogenic molecules. Vaccine 12:1160-1166.

39. Ma, J. K.-C., M. Hunjan, R. Smith, C. Kelly and T. Lehner. 1990. An investigation into the mechanism of protection by local passive immunization with MAbs against Streptococcus mutans. Infect. Immun. 58:3407-3414.

40. Marples, R. R., D. C. E. Speller and B. D. Cookson. 1995. Prevalence of mupirocin resistance in Staphylococcus aureus. J. Hosp. Infect. 29:153-155.

41. Martin, J., F. Perdreau-Remington, M. Kartalija, O. Pasi, M. Webb, J. Gerberding, H. Chambers, M. Tauber and B. Lee. 1999. A randomized clinical trial of mupirocin in the eradication of Staphylococcus aureus nasal carriage in human immunodeficiency virus disease. J. Infect. Dis. 180:896-899.

42. Martin, R. R. and A. White. 1967 The Selective Activity of Lysostaphin in Vivo. J. Lab. and Clin. Med. 70:1-8.

43. Mazenec, M. B., C. S. Kaetzel, M. E. Lamm, D. Fletcher, J. Peterra and J. G. Nedrud. 1995. Intracellular neutralization of Sendai and influenza viruses by IgA MAbs. Adv. Exp. Med. Biol. 371A:651-654.

44. McDevitt, D., P. Francois, P. Vaudaux and T. Foster. 1994. Molecular characterization of the clumping factor (fibrinogen receptor) of Staphylococcus aureus. Mol. Microbiol. 11:237-248.

45. Merkus, F. W., J. C. Verhoef, N. G. Schipper, and E. Marttin. 1999. Cyclodextrins in nasal drug delivery. Advan. Drug Deliv. Rev. 36:41-57.

46. Mest, D. R., D. H. Wong, K. J. Shimoda, M. E. Mulligan and S. E. Wilson. 1994. Nasal colonization with methicillin-resistant Staphylococcus aureus on admission to surgical intensive care unit increases the risk of infection. Anesth. Analg. 78:644-650.

47. Mosher, D. F. and R. A. Proctor. 1980. Binding and factor XIII-mediated cross linking of a 27-kilodalton fragment of fibronectin to Staphylococcus aureus. Science 209:927-929.

48. Natsume, H., S. Iwata, K. Ohtak, M. Miyamoto, M. Yamaguchi, K. Hosoya, and D. Kobayashi. 1999. Screening of cationic compounds as an absorption enhancer for nasal drug delivery. Int. J. Pharma. 185:1-12.

49. Nealon, T. J. and S. J. Mattingly. 1984. Role of cellular lipoteichoic acids in mediating adherence of serotype III strains pf group B streptococci to human embryonic, fetal and adult epithelial cells. Infect. Immun. 43:523-530.

50. Nelson, M. and M. McClelland. 1992. Use of DNA methyltransferase/endonuclease enzyme combinations for megabase mapping of chromosomes. Methods Enzymol. 216:279-303.

51. Nguyen, M. H., C. Kauffman, R. Goodman, C. Squier, R. Arbeit, N. Singh, M. Wagener and V. Yu. 1999. Nasal carriage of and infection with Staphylococcus aureus in HIV-infected patients. Ann. Int. Med. 130:221-225.

52. Olmsted, S. and N. Norcross. 1992. Effect of specific antibody on adherence of Staphylococcus aureus to bovine mammary epithelial cells. Infect. Immun. 60:249-256.

53. Patrick, C. C., M. R. Plaunt, S. V. Hetherington and S. M. May. 1992. Role of the Staphylococcus epidermidis slime layer in experimental tunnel tract infections. Infect. Immun. 60:1363-1367.

54. Pei, L., M. Palma, M. Nilsson, B. Guss and J.-I. Flock. 1999. Functional studies of fibrinogen binding protein from Staphylococcus epidermidis. Infect. Immun. 67:4525-4530.

55. Pennington, J. 1990. Newer uses for intravenous immunoglobulins as anti-infective agents. Antimicrob. Agents Chemother. 34:1463-1466.

56. Procter, R. A., R. J. Hamill, D. F. Mosher, J. A. Textor and P. J. Olbrantz. 1983. Effects of subinhibitory concentrations of antibiotics on Staphylococcus aureus interactions with fibronectin. J. Antimicro. Chemo. 12:85-95.

57. Quickel, K. E., Jr., R. Selden, J. R. Caldwell, N. F. Nora, and W. Schaffner. 1971. Efficacy and Safety of Topical Lysostaphin Treatment of Persistent Nasal Carriage of Staphylococcus aureus. App. Microbiol. 22:446-450.

58. Ramisse, F., M. Szatanik, P. Binder and J.-M. Alonso. 1993. Passive local immunotherapy of experimental staphylococcal pneumonia with human intravenous immunoglobulin. J. Infect. Dis. 168:1030-1033.

59. Sambrook et al. 1989. Molecular Cloning, A Laboratory Manual, 2^{nd} Ed., Cold Spring Harbor Press, Cold Spring Harbor, N.Y.

60. Sanford, B. A., V. L. Thomas and M. A. Ramsay. 1989. Binding of staphylococci to mucus in vivo and in vitro. Infect. Immun. 57:3735-3742.

61. Schennings, T., A. Heimdahl, K. Coster and J. Flock. 1993. Immunization with fibronectin binding protein from Staphylococcus aureus protects against experimental endocarditis in rats. Microb. Pathog. 15:227-236.

62. Schwab, U. E., A. E. Wold, J. L. Carson, M. W. Leigh, P.-W. Cheng, P. H. Gilligan and T. F. Boat. 1993. Increased adherence of Staphylococcus aureus from cystic fibrosis lungs to airway epithelial cells. Am. Rev. Respir. Dis. 148:365-369.

63. Soto, N., A. Vaghjimal, A. Stahl-Avicolli, J. Protic, L. Lutwick and E. Chapnick. 1999. Bacitracin versus mupirocin for Staphylococcus aureus nasal colonization. Infect. Cont. Hosp. Epidem. 20:351-353.

64. Suzuki, Y. and Y. Makino. 1999. Mucosal drug delivery using cellulose derivative as a functional polymer. J. Control. Release. 62:101-107.

65. Takenaga, M., Y. Sirizawa, Y. Azechi, A. Ochiai, Y. Kosaka, R. Igarashi, and Y. Mizushima. 1998. Microparticle resins as a potential nasal drug delivery system for insulin. J. Control. Release. 52:81-87.

66. Teti, G., M. S. Chiofalo, F. Tomasello, C. Fava and P. Mastroeni. 1987. Mediation of Staphylococcus saprophyticus adherence to uroepithelial cells by lipoteichoic acid. Infect. Immun. 55:839-842.

67. Thumm, G. and F. Gotz. 1997. Studies on prolysostaphin processing and characterization of the lysostaphin immunity factor (Lif) of Staphylococcus simulans biovar staphylolyticus. Mol. Microbiol. 23:1251-65.

68. VadenBergh, M., E. Yzerman, A. Van Belkum, H. Boelens, M. Simmons and H. Verbrugh. 1999. Follow-up of Staphylococcus aureus nasal carriage after 8 years: redefining the persistent carrier state. J. Clin. Micro. 37:3133-3140.

69. Vaudaux, P., E. Huggler, P. Lerch, J. Morgenthaler, U. Nydegger, F. Schumacher-Perdreau, P. Lew and F. Waldvogel. 1989. Inhibition by immunoglobulins of Staphylococcus aureus adherence to fibronectin-coated foreign surfaces. J. Invest. Surg. 2:397-408.

70. Ward, T. T. 1992. Comparison of in vitro adherence of methicillin-sensitive and methicillin-resistant Staphylococcus aureus to human nasal epithelial cells. J. Infect. Dis. 166:400-404.

71. White, A. and J. Smith. 1963. Nasal reservoir as the source of extranasal staphylococci. Antimicrob. Agent. Chem. 3:679-683.

72. Yano, M., Y. Doki, M. Inoue, T. Tsujinaka, H. Shiozaki and M. Monden. 2000. Preoperative intranasal mupirocin ointment significantly reduces postoperative infection with Staphylococcus aureus in patients undergoing upper gastrointestinal surgery. Surg. Today (Japan). 30:16-21.

73. Yokoyama, Y., Y. Harabuchi, H. Kodama, H. Murakata and A. Kataura. 1996. Systemic immune response to Streptococcal and Staphylococcal lipoteichoic acids in children with recurrent tonsillitis. Acta Otolaryngol. Suppl. (Norway). 523:108-111.

74. Yu, V. L., A. Goetz, M. Wagener, P. B. Smith, J. D. Rihs, J. Hanchett and J. J. Zuravleff. 1986. Staphylococcus aureus nasal carriage and infection in patients on hemodialysis. New Engl. J. Med. 315:91-96.

75. Zygmunt WA and P.A. Tavormina. 1972 Lysostaphin: model for a specific enzymatic approach to infectious disease. Progr. Drug Res. 16:309-333.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A lysostaphin intranasal composition comprising a) a viscous formulation and b) at least 0.125% lysostaphin.

2. The composition of claim 1, comprising 0.125%-10% lysostaphin.

3. The composition of claim 1, wherein the lysostaphin is selected from any wild-type lysostaphin, human or animal lysostaphin, lysostaphin mutant, variant or fragment, synthetic lysostaphin, or recombinantly expressed lysostaphin; wherein the lysostaphin has proteolytic activity against glycine-containing bridges in the cell wall peptidoglycan of staphylococci.

4. The composition of claim 1, further comprising at least one antibiotic, antibacterial agent, or staphyolytic enzyme other than lysostaphin.

5. The composition of claim 1, further comprising bacitracin.

6. The composition of claim 1, further comprising phi11 hydrolase.

7. The composition of claim 1, wherein the viscous formulation is a viscous liquid formulation.

8. The composition of claim 1, wherein the viscous formulation is a cream formulation.

9. The composition of claim 1, comprising a cream base, at least one consistency regulator, at least one emulsifier, and at least one stabilizer.

10. The composition of claim 1, comprising at least one of petrolatum, SOFTISAN 649, paraffin, beeswax, MIGLYOL 812, zinc stearate, and aluminum stearate (metal stearate).

11. The composition of claim 1, comprising about: 15%-50% MIGLYOL 812, 15%-50% SOFTISAN 649, 15%-50% White Petrolatum, 0%-10% Paraffin, 0%-10% Beeswax, and 0%-5% Aluminum Stearate, and 0%-5% Zinc or aluminum Stearate (metal stearate).

12. The composition of claim 1, comprising 36% MIGLYOL 812 (Caprylic/Capric Triglyceride), 24.2% SOFTISAN 649 (Bis-Diglyceryl Polyacyladipate-2), 27.5% white petrolatum, 3.4% paraffin, 3.4% beeswax, and 0.5% zinc or aluminum stearate (metal stearate).

13. The composition of claim 1, comprising at least one of polyphosphoesters, polyethylene glycol, and high molecular weight poly (lactic acid), hydroxypropyl cellulose, chitosan, and polystyrene sulfanate.

14. The composition of claim 1, comprising at least one microencapsulating agent.

15. The composition of claim 1, comprising at least one of polystyrene sulfonate and chitosan.

16. The composition of claim 1, comprising about 0.5% polystyrene sulfonate.

17. A method for treating a patient comprising, administering to the nose of a human or non-human patient via one or more instillations, an effective amount of a composition comprising a) a viscous formulation and b) at least 0.125% lysostaphin.

18. The method of claim 17, wherein the composition is instilled into the patient's nose by any of: swabbing with a finger or applicator; squeezing a tube, syringe, or applicator of the composition into the nares; via aerosol, mist, nasal spray, or nasal drops.

19. The method of claim 17, wherein administration results in a beneficial outcome selected from one or more of:
a) no nasal colonization by staphylococci for at least 12 hours after a final instillation of the composition,
b) a discernable decrease in the number of staphylococcal colonies in the nares within a time after a final instillation of the composition, wherein said time is selected from within 4 hours, within 12 hours, and within 24 hours after a final instillation,
c) a decrease in the frequency of positive cultures taken from the nares within a time after a final instillation of the composition, wherein said time is selected from within 4 hours, within 12 hours, and within 24 hours after a final instillation;
d) continued activity of the lysostaphin in the nares for a time after a final instillation of the composition, wherein said time is selected from at least 8 hours, at least 12 hours, at least 12 hours, and at least 48 hours after a final instillation;
e) eradication, alleviation, or blockage of colonization of the patient's nares by staphylococci by a single dose of the composition;
f) eradication, alleviation, or blockage of colonization of the patient's nares by staphylococci by up to 10 instillations of the composition;
g) a blocking or prophylaxis against future staphylococcal colonization in the patient's nares; and
h) any discernable reduction in the likelihood of staphylococcal infection in the patient.

20. The method of claim 19, wherein the staphylococci are S. aureus.

21. The method of claim 17, wherein the composition comprises 0.125%-10% lysostaphin.

22. The method of claim 17, wherein the lysostaphin is selected from any wild-type lysostaphin, human or animal lysostaphin, lysostaphin mutant, variant or fragment, synthetic lysostaphin, or recombinantly expressed lysostaphin; wherein the lysostaphin has proteolytic activity against glycine-containing bridges in the cell wall peptidoglycan of staphylococci.

23. The method of claim 17, wherein the composition further comprises at least one antibiotic, antibacterial agent, or staphyolytic enzyme other than lysostaphin.

24. The method of claim 17, wherein the composition further comprises bacitracin.

25. The method of claim 17, wherein the composition further comprises phi11 hydrolase.

26. The method of claim 17, wherein the viscous formulation is a viscous liquid formulation.

27. The method of claim 17, wherein the viscous formulation is a cream formulation.

28. The method of claim 17, wherein the viscous formulation comprises about: 15%-50% MIGLYOL 812, 15%-50% SOFTISAN 649, 15%-50% White Petrolatum, 0%-10% Paraffin, 0%-10% Beeswax, and 0%-5% Aluminum Stearate, and 0%-5% Zinc or aluminum Stearate (metal stearate).

29. The method of claim 17, wherein the viscous formulation comprises 36% MIGLYOL 812 (Caprylic/Capric Triglyceride), 24.2% SOFTISAN 649 (Bis-Diglyceryl Polyacyladipate-2), 27.5% white petrolatum, 3.4% paraffin, 3.4% beeswax, and 0.5% zinc or aluminum stearate (metal stearate).

30. The method of claim 17, wherein the composition comprises at least one of polyphosphoesters, polyethylene glycol, and high molecular weight poly (lactic acid), hydroxypropyl cellulose, chitosan, and polystyrene sulfanate.

31. The method of claim 17, wherein the composition comprises, comprising at least one microencapsulating agent.

32. The method of claim 17, wherein the composition comprises at least one of polystyrene sulfonate and chitosan.

33. The method of claim 17, wherein the composition comprises about 0.5% polystyrene sulfonate.

34. A method for reducing the emergence of lysostaphin resistant bacteria in a population of patients comprising administering the composition of claim 1 to the nares of patients in that population.
